(19) United States
(12) Patent Application Publication
(10) Pub. No.: US 2013/0345231 A1
(43) Pub. Date: Dec. 26, 2013

(54) ANTICANCER THERAPEUTIC AGENTS

(71) Applicants: Robert J. Hickey, Lakeview Terrace, CA (US); Linda H. Malkas, Lakeview Terrace, CA (US)

(72) Inventors: Robert J. Hickey, Lakeview Terrace, CA (US); Linda H. Malkas, Lakeview Terrace, CA (US)

(73) Assignee: INDIANA UNIVERSITY RESEARCH AND TECHNOLOGY CORPORATION, Indianapolis, IN (US)

(21) Appl. No.: 14/004,239

(22) PCT Filed: Mar. 21, 2012
(86) PCT No.: PCT/US12/29972

§ 371 Date: Sep. 10, 2013

Related U.S. Application Data
(60) Provisional application No. 61/466,508, filed on Mar. 23, 2011.

(51) Int. Cl. A61K 31/519; A61K 31/403; A61K 31/47; A61K 31/517; A61K 31/407; A61K 31/506; A61K 45/06; A61K 31/4468

(52) U.S. Cl. CPC A61K 31/519; A61K 45/06; A61K 31/403; A61K 31/4468; A61K 31/517; A61K 31/407; A61K 31/506; A61K 31/47
USPC 514/252.14; 514/411; 514/329; 514/266.3; 514/412; 514/311; 514/267

(57) ABSTRACT

The invention described herein pertains to anticancer therapeutic agents that exhibit preferential cytotoxicity to malignant cells that express a cancer specific isoform of proliferating cell nuclear antigen (caPCNA) compared to cytotoxicity to comparable non-malignant cells, pharmaceutical compositions comprising the agents, and their use in cancer therapy.

This application claims the benefit of U.S. provisional application 61/466,508, filed 23 Mar. 2011, which is incorporated herein by reference in its entirety.

### GOVERNMENT RIGHTS

This invention was made with government support under Grant No. W81XWH-07-1-0707 awarded by the Congressionally Directed Medical Research Programs (CDMRP) Breast Cancer Research Program of the Department of Defense. The U.S. government has certain rights in the invention.

### TECHNICAL FIELD

The invention described herein pertains to anticancer therapeutic agents that exhibit preferential cytotoxicity to malignant cells that express a cancer specific isoform of proliferating cell nuclear antigen (caPCNA) compared to cytotoxicity to comparable non-malignant cells, pharmaceutical compositions comprising the agents, and their use in cancer therapy.

### BACKGROUND AND SUMMARY OF THE INVENTION

Proliferating cell nuclear antigen (PCNA) plays an important role in the process of DNA replication, repair, chromosomal recombination, cell cycle check-point control and other cellular proliferative activities. In conjunction with an adaptor protein, replication factor C(RFC), PCNA forms a moving clamp that is the docking point for DNA polymerases delta and epsilon. Different isoforms of proliferating cell nuclear antigen (PCNA) that display both acidic and basic isoelectric points (pI) have been demonstrated. Analysis of PCNA by two-dimensional polyacrylamide gel electrophoresis (2D PAGE) from both malignant and non-malignant breast cells (referred to as non-malignant PCNA or nmPCNA) and tissues revealed the presence of an acidic form of PCNA only in malignant cells (referred to as the cancer-specific PCNA or csPCNA or caPCNA, herein caPCNA). This difference in isoelectric point between these two forms of PCNA appears to result from an alteration in the ability of the malignant cells to post-translationally modify the PCNA polypeptide and is not due to a genetic change within the PCNA gene.

It has been shown that antibodies or peptides which bind only to the caPCNA isoform and not to the nmPCNA isoform interfere with intracellular protein-protein interactions, thereby causing a reduction in the proliferative potential of cancer. See, for example, WO 2006/116631 and WO 2007 098/415.

Also, PCNA is also known to interact with other factors like FEN-1, DNA ligase, and DNA methyl transferase. Additionally, PCNA was also shown to be an essential player in multiple DNA repair pathways. Interactions with proteins like the mismatch recognition protein, Msh2, and the nucleotide excision repair endonuclease, XPG, have implicated PCNA in processes distinct from DNA synthesis. Interactions with multiple partners generally rely on mechanisms that enable PCNA to selectively interact in an ordered and energetically favorable way.

We have discovered small molecule therapeutic agents which exhibit preferential cytotoxicity to malignant cells that express the cancer specific isoform of proliferating cell nuclear antigen (caPCNA) compared to cytotoxicity to comparable non-malignant cells. Without being bound by theory and as described below, it is believed these small molecule therapeutic agents exert their action through specific binding modes which inhibit protein-protein interactions involving caPCNA. Once docked with caPCNA, these molecules either reduce or prevent caPCNA from interacting with its natural set of binding partners. This disruption in binding partner interaction results in inhibition of specific cellular functions requiring both caPCNA and its binding partner (e.g., DNA replication and DNA repair). See, for example, FIG. 1.

Thus, small molecules bound to the protein-protein interaction domain of caPCNA or its binding partners (including, but not limited to, DNA polymerase δ, Xeroderma Pigmentosum G protein (XPG), or Flap-endonuclease (FEN-1)), would in-turn reduce/eliminate the ability of a cancer cell to properly replicate and/or repair its DNA; leading to the killing of the cancer cell. Also, the small molecule inhibitors of caPCNA-mediated function might have better therapeutic efficacy than the caPCNA derived octapeptides described above, because of the intrinsic stability properties of these specific small molecules within the blood-stream and tissues, relative to the stability of the peptides, and the issue of selectively directing sufficient quantities of the peptide into cancer cells without having the bulk of the peptide being taken up by cells in the blood-stream or surrounding tissues.

In one illustrative embodiment of the invention, a method of reducing cellular proliferation of malignant cells that express a cancer specific isoform of proliferating cell nuclear antigen (caPCNA) in a patient in need thereof, comprising administering a therapeutically effective amount of a compound of the formula

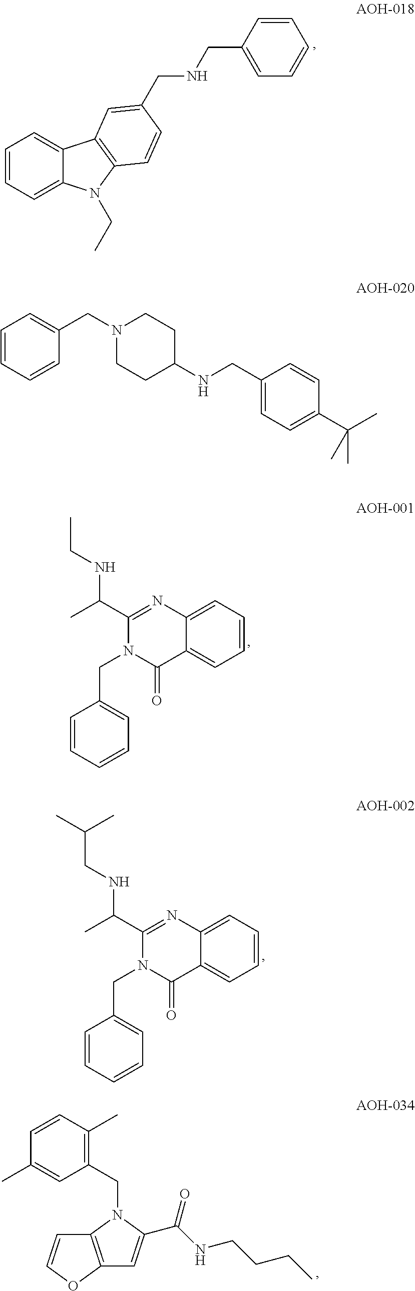

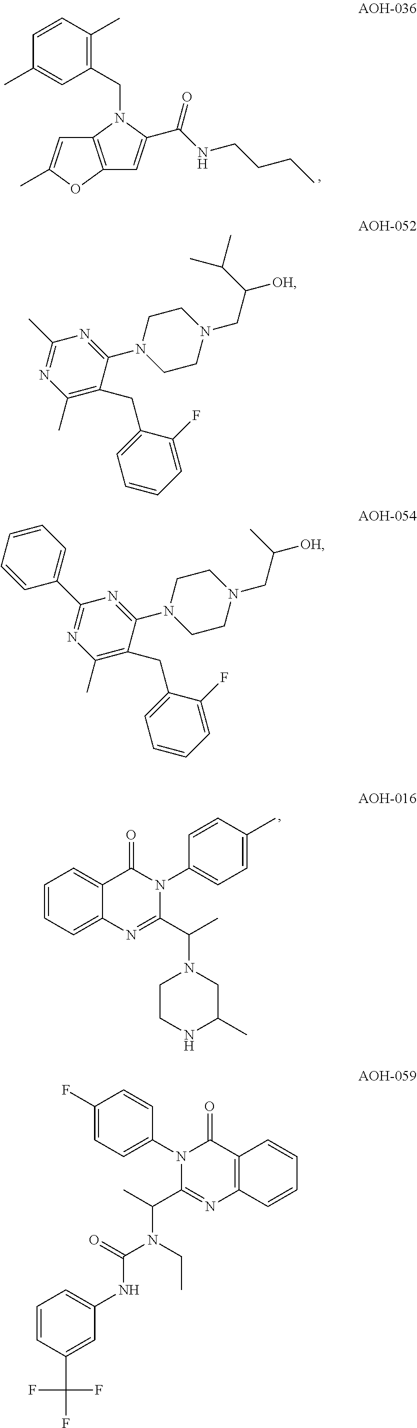

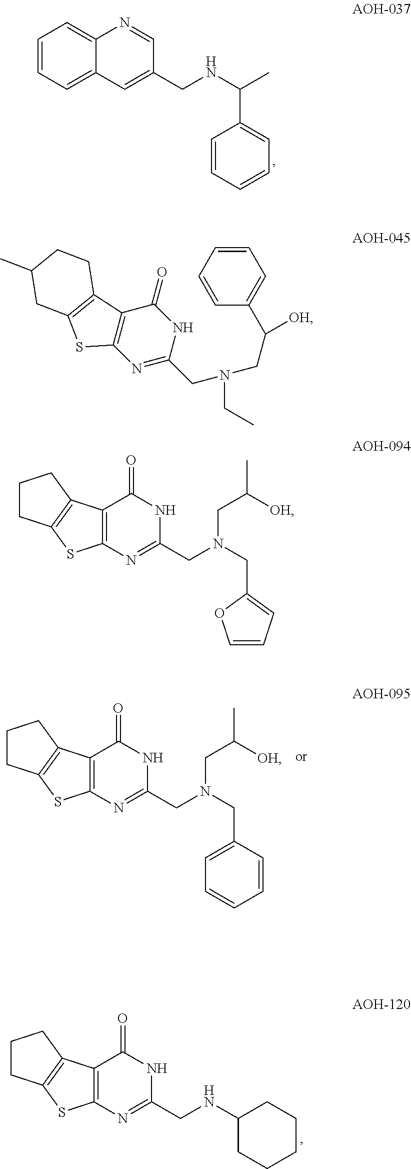

or a substituted derivative thereof, or a pharmaceutically acceptable salt thereof, is described herein.

In another embodiment, there is described the use of a compound as described above or a substituted derivative thereof, or a pharmaceutically acceptable salt thereof, for reducing cellular proliferation of malignant cells that express a cancer specific isoform of proliferating cell nuclear antigen (caPCNA).

In another embodiment, there is described a pharmaceutical composition comprising a compound as described above or a substituted derivative thereof, or a pharmaceutically acceptable salt thereof, and further comprising one or more carriers, diluents, or excipients, or a combination thereof.

It is appreciated herein that the compounds described herein may be used alone or in combination with other compounds useful for treating cancer, including those compounds that may be therapeutically effective by the same or different modes of action.

### BRIEF DESCRIPTION OF THE DRAWINGS

FIG. 1: Proposed Scheme for caPCNA action. Panel A represents how doxorubicin (DOX) induced DNA damage is normally repaired in cancer cells. caPCNA would interact with DNA repair proteins to facilitate fixing the damaged DNA. Panel B represents the conditions when the small molecule therapeutic agent (SM) is present in a cell that has DOX induced DNA damage. In this case the small molecule therapeutic agent (SM) binding with caPCNA or its binding partner competes with the full length caPCNA protein binding to its DNA repair protein partners, thereby, preventing the repair of the damaged DNA.

FIG. 2A-C: Identification of compounds exhibiting differential cytotoxicity toward malignant and non-malignant breast cells. Exponentially growing malignant (MCF-7) and non-malignant (MCF-10A) breast cells were incubated for 72 hours with 100 μM of the indicated compounds in growth media, before cell viability was determined colorimetrically using the MTT assay. The Y-axis shows relative cell viability at the end of the incubation with the compounds. Relative viability was determined by comparison to the viability of the cell cultures incubated in the presence of phosphate buffered saline/dimethylsulfoxide (PBS/DMSO) (vehicle) control instead of compound.

FIG. 3: Viability of MCF7 and MCF10A cells following a 72 hour incubation with 10 μM of those compounds previously exhibiting preferential cytotoxicity at 100 μM toward breast cancer cells. Exponentially growing cultures of MCF7 and MCF-10A cells were incubated for 72 hours with the compounds indicated, and relative viability was determined colorimetrically using the MTT assay. Viability was determined relative to control cultures incubated with PBS/DMSO in place of compound.

FIG. 4: Correlation between AOH mediated cytotoxicity in MCF7 cells, and in vitro DNA replication activity mediated by the DNA synthesome isolated from these cells. Cell viability was determined colorimetrically using the MTT assay following a 72 hour incubation with 10 μM of the compound indicated in the figure. Percent viability was determined relative to cultures containing PBS/DMSO in place of compound. In vitro DNA replication activity was determined using the standard T-antigen and SV40 origin dependent in vitro DNA replication reaction (L. Malkas et al., Biochemistry, 29, 6362-6374 (1990)), and percent inhibition was determined relative to a reaction containing PBS in place of compound.

FIG. 5: MCF7 cell extracts were incubated with AOH-37 or PBS/DMSO prior to incubating the cell extracts with anti-XP-G antibody, and collecting the antibody bound protein complexes by affinity chromatography using Protein G agarose beads. The captured antibody and antibody bound proteins were eluted from the Protein-G beads by heating them for 5 minutes at 95° C. in SDS denaturing gel loading buffer, and the released proteins were resolved by electrophoresis through a SDS 8% polyacrylamide gel. The resolved proteins were transferred to a PVDF filter membrane, and the membrane probed with anti-PCNA antibody. The location on the filter and the relative abundance of the co-precipitated PCNA was identified by chemilluminescense.

FIG. 6: Cytotoxicity AOH-45 toward malignant and non-malignant breast cells. Exponentially growing MCF7 and MCF10A cells were incubated for 72 hours with AOH-45, or a PBS/DMSO control, and cell viability was determined for the drug treated cells relative to the control cultures using the MTT assay. The effect an equivalent amount of DMSO had on cell viability for each drug concentration used was also determined, and viability was determined relative to cultures receiving no DMSO.

FIG. 7: Relative cytotoxicity of the AOH compounds docking with caPCNA toward cultured Panc-1 (A) and Paca2 (B) cells. Relative cytotoxicity was determined by a colorimetric assay (MTT) measuring cell viability following a 72 hour incubation with the compounds indicated. Cells were exposed to DMSO/saline (series 1) and 12.5 (series 2) 25 (series 3) and 50 (series 4) μM of the indicated drug dissolved in DMSO for 72 hours prior to performing the MTT assay.

FIG. 8: 50 μM AOH-18, AOH-20, and AOH-39 were individually incubated with exponentially growing Panc-1 or Paca-2 cells for 72 hours, after which cell viability was determined colorimetrically using MTT.

FIG. 9: Comparative effect of AOH-39 on the viability of stimulated normal peripheral blood mononucleocytes and pancreatic cancer cell lines. PBMC, Panc-1, and Paca-2 cells were incubated with the indicated concentrations of AOH-39 for 72 hours, prior to determining cell viability using the MTT assay. Cell viability was determined as a percentage of control cultures receiving PBS/DMSO in place of AOH-39.

FIG. 10: Differential cytotoxic response of breast cancer versus pancreatic cancer cells following incubation with AOH-18. Exponentially growing cells were exposed for 72 hours to AOH-18 and viability determined using the MTT assay. Cell viability was determined relative to drug free control cultures which contained PBS/DMSO in place of compound.

FIG. 11: Inhibition of breast cancer cell viability following incubation with various AOH compounds. Exponentially growing MCF-7 cells were incubated for 72 hours with the compounds indicated, and cell viability determined colorimetrically using the MTT assay. Viability of drug exposed cultures was determined relative to a no drug control, (receiving PBS/DMSO in place of compound).

FIG. 12: Dose response curves evaluating the cytotoxic response of malignant (MCF7) and non-malignant (MCF10A) breast cells to molecules binding Fen1. Exponentially growing cells were incubated for 72 hours with the concentration of the drug indicated in each figure, and cell viability was determined using the MTT assay. LC50 values in the MCF7 cells were determined for each of the 3 compounds examined, and the effect of this concentration on the viability of MCF10A (non-malignant) cells was determined. Viability at each of the drug concentrations examined was determined relative to that of the control cultures containing PBS in place of compound.

FIG. 16: AOH-95 inhibits origin dependent in vitro DNA replication. AOH-95 was pre-incubated with MCF7 cell extract containing the partially purified DNA synthesome for 15 minutes prior to initiating the in vitro DNA synthetic reaction. AOH-95 mediated inhibition of the replication reaction was reported as a percentage of the reaction performed in the absence of compound.

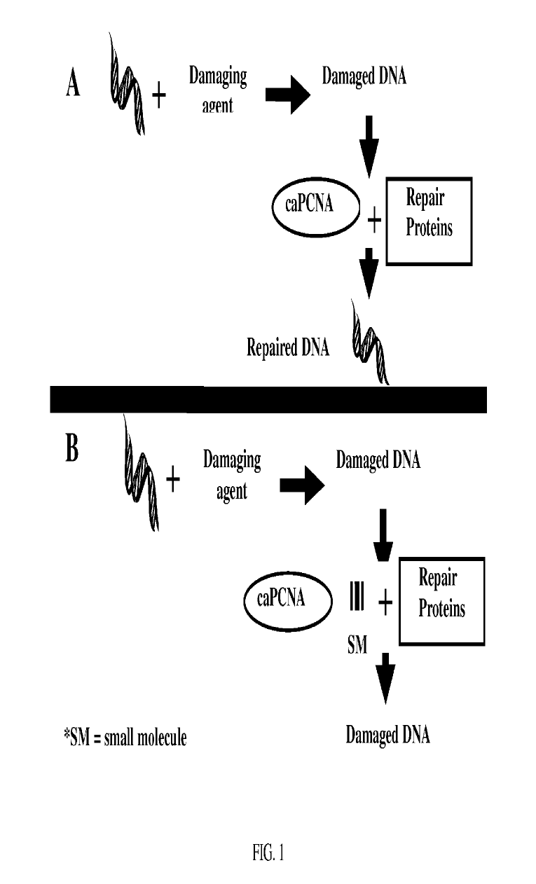

### DETAILED DESCRIPTION

Embodiments of the invention are further described by the following enumerated clauses:

1. A method of reducing cellular proliferation of malignant cells that express a cancer specific isoform of proliferating cell nuclear antigen (caPCNA) in a patient in need thereof, comprising administering a therapeutically effective amount of a compound of the formula

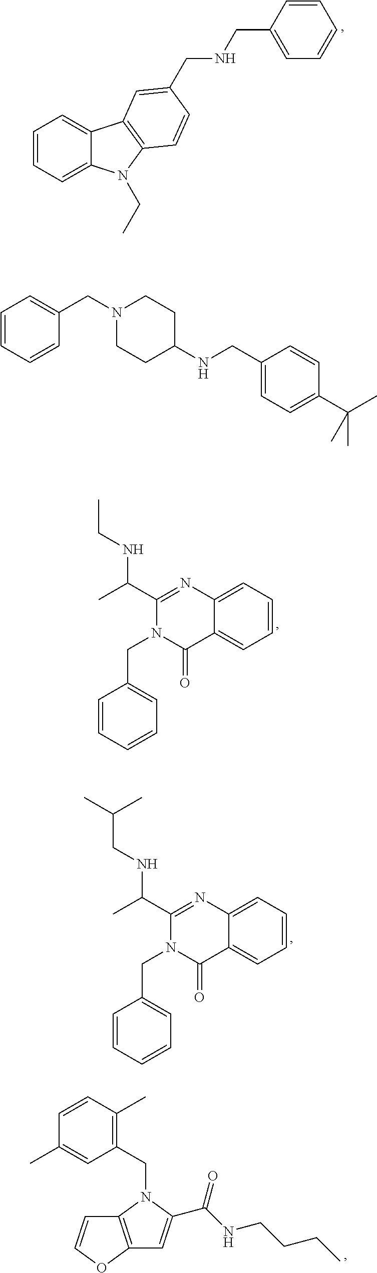

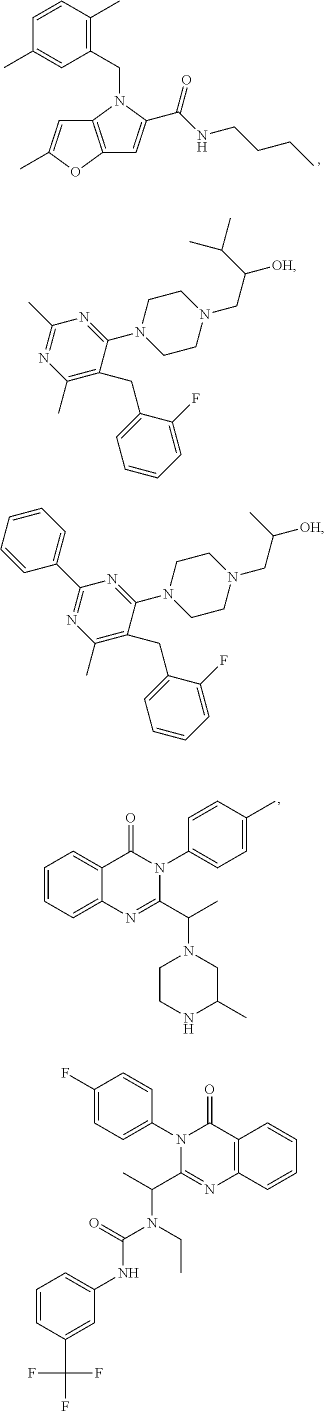

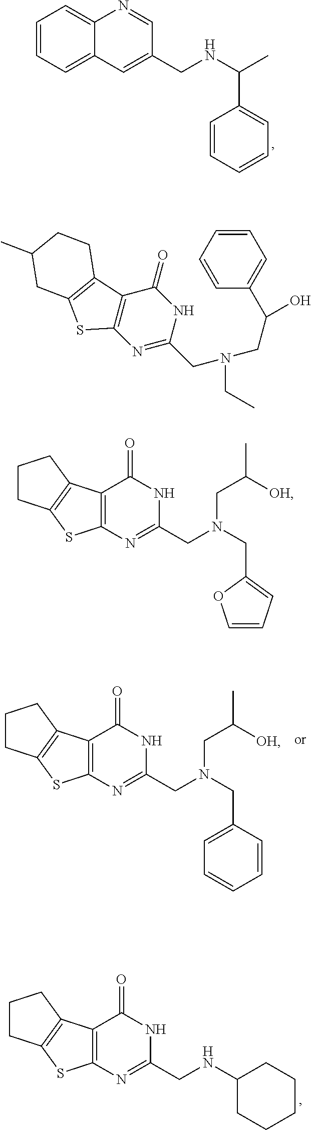

or a substituted derivative thereof, or a pharmaceutically acceptable salt thereof.

2. Use of a compound as described in clause 1 or a substituted derivative thereof, or a pharmaceutically acceptable salt thereof, for reducing cellular proliferation of malignant cells that express a cancer specific isoform of proliferating cell nuclear antigen (caPCNA).

3. A pharmaceutical composition comprising a compound as described in clause 1 or a substituted derivative thereof, or a pharmaceutically acceptable salt thereof, and further comprising one or more carriers, diluents, or excipients, or a combination thereof.

4. The method, use or composition of any of clauses 1-3 wherein the compound is

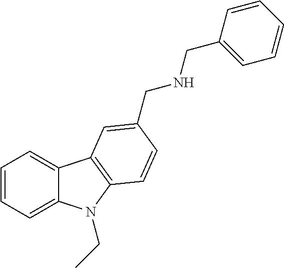 [SMILES: CCn1c2ccccc2c2cc(CNCc3ccccc3)ccc21]

or a substituted derivative thereof, or a pharmaceutically acceptable salt thereof.

5. The method, use or composition of any of clauses 1-3 wherein the compound is

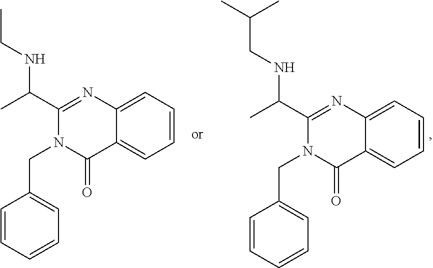 [SMILES: CCNC(C)c1nc2ccccc2c(=O)n1Cc1ccccc1 ; CC(C)CNC(C)c1nc2ccccc2c(=O)n1Cc1ccccc1]

or a substituted derivative thereof, or a pharmaceutically acceptable salt thereof.

6. The method, use or composition of any of clauses 1-3 wherein the compound is

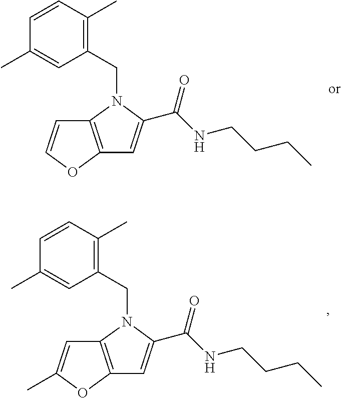

or a substituted derivative thereof, or a pharmaceutically acceptable salt thereof.

7. The method, use or composition of any of clauses 1-3 wherein the compound is

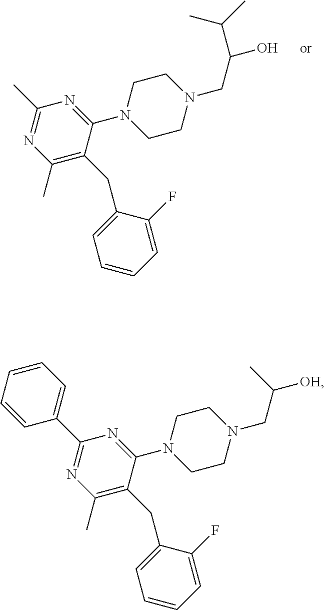 [SMILES: Cc1nc(C)c(Cc2ccccc2F)c(N2CCN(CC(O)C(C)C)CC2)n1 ; Cc1nc(-c2ccccc2)nc(N2CCN(CC(C)O)CC2)c1Cc1ccccc1F]

or a substituted derivative thereof, or a pharmaceutically acceptable salt thereof.

8. The method, use or composition of any of clauses 1-3 wherein the compound is

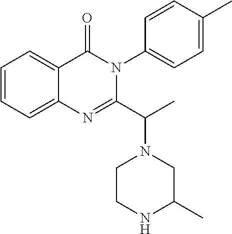 [SMILES: Cc1ccc(-n2c(C(C)N3CCNC(C)C3)nc3ccccc3c2=O)cc1]

or a substituted derivative thereof, or a pharmaceutically acceptable salt thereof.

9. The method, use or composition of any of clauses 1-3 wherein the compound is

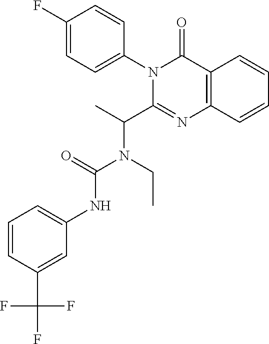 [SMILES: CCN(C(=O)Nc1cccc(C(F)(F)F)c1)C(C)c1nc2ccccc2c(=O)n1-c1ccc(F)cc1]

or a substituted derivative thereof, or a pharmaceutically acceptable salt thereof.

10. The method, use or composition of any of clauses 1-3 wherein the compound is

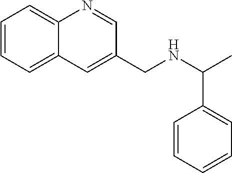 [SMILES: CC(NCc1cnc2ccccc2c1)c1ccccc1]

or a substituted derivative thereof, or a pharmaceutically acceptable salt thereof.

11. The method, use or composition of any of clauses 1-3 wherein the compound is

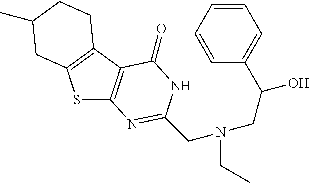 [SMILES: CCN(Cc1nc2sc3c(c2c(=O)[nH]1)CCC(C)C3)CC(O)c1ccccc1]

or a substituted derivative thereof, or a pharmaceutically acceptable salt thereof.

12. The method, use or composition of any of clauses 1-3 wherein the compound is

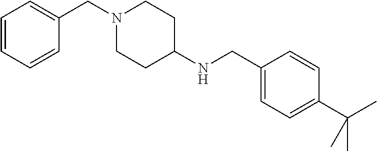 [SMILES: CC(C)(C)c1ccc(CNC2CCN(Cc3ccccc3)CC2)cc1]

or a substituted derivative thereof, or a pharmaceutically acceptable salt thereof.

13. The method, use or composition of any of clauses 1-12 wherein the cancer is breast cancer.

14. The method, use or composition of any of clauses 1-12 wherein the cancer is pancreatic cancer.

15. The method or use of any of clauses 1-2 or 4-14 wherein the use is to augment another chemotherapeutic method.

16. A pharmaceutical composition comprising a compound as described in any of clauses 1 and 4-12 and a further chemotherapeutic agent.

As used herein, a substituted derivative of an illustrated compound includes one in which one or more hydrogens has been replaced by, for example, a halo, hydroxy and derivatives thereof, amino and derivatives thereof, thio and derivatives thereof, acyl, alkyl, alkenyl, alkynyl, cycloalkyl, aryl, arylalkyl, heteroalkyl, cycloheteroalkyl, heteroaryl, heteroarylalkyl, alkylsulfinyl, alkylsulfonyl, arylsulfinyl, arylsulfonyl, heteroarylsulfinyl or heteroarylsulfonyl group, each of which may bear one or more substituents, as well as a derivative in which, for example one or more halo, hydroxy or alkyl groups has been replaced by a hydrogen.

In each of the foregoing and following embodiments, it is to be understood that the formulae include and represent not only all pharmaceutically acceptable salts of the compounds, but also include any and all hydrates and/or solvates of the compound formulae. It is appreciated that certain functional groups, such as the hydroxy, amino, and like groups form complexes and/or coordination compounds with water and/or various solvents, in the various physical forms of the compounds. Accordingly, the above formulae are to be understood to include and represent those various hydrates and/or solvates. In each of the foregoing and following embodiments, it is also to be understood that the formulae include and represent each possible isomer, such as stereoisomers and geometric isomers, both individually and in any and all possible mixtures. In each of the foregoing and following embodiments, it is also to be understood that the formulae include and represent any and all crystalline forms, partially crystalline forms, and non crystalline and/or amorphous forms of the compounds.

Illustrative derivatives include, but are not limited to, both those compounds that may be synthetically prepared from the compounds described herein, as well as those compounds that may be prepared in a similar way as those described herein, but differing in the selection of starting materials. For example, described herein are compounds that include aromatic rings. It is to be understood that derivatives of those compounds also include the compounds having for example different substituents on those aromatic rings than those explicitly set forth in the definition above. In addition, it is to be understood that derivatives of those compounds also include the compounds having those same or different functional groups at different positions on the aromatic ring. Similarly, derivatives include variations of other substituents on the compounds described herein, such as on an alkyl group or an amino group, and the like.

It is to be understood that such derivatives may include prodrugs of the compounds described herein, compounds described herein that include one or more protection or protecting groups, including compounds that are used in the preparation of other compounds described herein.

Illustrative derivatives include, but are not limited to, those compounds that share functional and in some cases structural similarity to those compounds described herein. For example, described herein are compounds that include a ring system. Illustrative substituted derivatives include, but are not limited to, the corresponding ring expanded compounds, and the corresponding ring systems that include one or more heteroatoms, such as by substitution of a methylene group with an oxa, thia or optionally substituted amino group, or substitution of an aromatic C—H group with an N.

The compounds described herein may contain one or more chiral centers, or may otherwise be capable of existing as multiple stereoisomers. It is to be understood that in one embodiment, the invention described herein is not limited to any particular stereochemical requirement, and that the compounds, and compositions, methods, uses, and medicaments that include them may be optically pure, or may be any of a variety of stereoisomeric mixtures, including racemic and other mixtures of enantiomers, other mixtures of diastereomers, and the like. It is also to be understood that such mixtures of stereoisomers may include a single stereochemical configuration at one or more chiral centers, while including mixtures of stereochemical configuration at one or more other chiral centers.

Similarly, the compounds described herein may include geometric centers, such as cis, trans, E, and Z double bonds. It is to be understood that in another embodiment, the invention described herein is not limited to any particular geometric isomer requirement, and that the compounds, and compositions, methods, uses, and medicaments that include them may be pure, or may be any of a variety of geometric isomer mixtures. It is also to be understood that such mixtures of geometric isomers may include a single configuration at one or more double bonds, while including mixtures of geometry at one or more other double bonds.

As used herein, the term “alkyl” includes a chain of carbon atoms, which is optionally branched. As used herein, the term “alkenyl” and “alkynyl” includes a chain of carbon atoms, which is optionally branched, and includes at least one double bond or triple bond, respectively. It is to be understood that alkynyl may also include one or more double bonds. It is to be further understood that in certain embodiments, alkyl is advantageously of limited length, including C₁-C₂₄, C₁-C₁₂, C₁-C₈, C₁-C₆, and C₁-C₄. It is to be further understood that in certain embodiments alkenyl and/or alkynyl may each be advantageously of limited length, including C₂-C₂₄, C₂-C₁₂, C₂-C₈, C₂-C₆, and C₂-C₄. It is appreciated herein that shorter alkyl, alkenyl, and/or alkynyl groups may add less lipophilicity to the compound and accordingly will have different pharmacokinetic behavior. Illustrative alkyl groups are, but not limited to, methyl, ethyl, n-propyl, isopropyl, n-butyl, isobutyl, sec-butyl, tert-butyl, pentyl, 2-pentyl, 3-pentyl, neopentyl, hexyl, heptyl, octyl and the like.

As used herein, the term “cycloalkyl” includes a chain of carbon atoms, which is optionally branched, where at least a portion of the chain in cyclic. It is to be understood that cycloalkylalkyl is a subset of cycloalkyl. It is to be understood that cycloalkyl may be polycyclic. Illustrative cycloalkyl include, but are not limited to, cyclopropyl, cyclopentyl, cyclohexyl, 2-methylcyclopropyl, cyclopentyleth-2-yl, adamantyl, and the like. As used herein, the term “cycloalkenyl” includes a chain of carbon atoms, which is optionally branched, and includes at least one double bond, where at least a portion of the chain in cyclic. It is to be understood that the one or more double bonds may be in the cyclic portion of cycloalkenyl and/or the non-cyclic portion of cycloalkenyl. It is to be understood that cycloalkenylalkyl and cycloalkylalkenyl are each subsets of cycloalkenyl. It is to be understood that cycloalkyl may be polycyclic. Illustrative cycloalkenyl include, but are not limited to, cyclopentenyl, cyclohexylethen-2-yl, cycloheptenylpropenyl, and the like. It is to be further understood that chain forming cycloalkyl and/or cycloalkenyl is advantageously of limited length, including C₃-C₂₄, C₃-C₁₂, C₃-C₈, C₃-C₆, and C₅-C₆. It is appreciated herein that shorter alkyl and/or alkenyl chains forming cycloalkyl and/or cycloalkenyl, respectively, may add less lipophilicity to the compound and accordingly will have different pharmacokinetic behavior.

As used herein, the term “heteroalkyl” includes a chain of atoms that includes both carbon and at least one heteroatom, and is optionally branched. Illustrative heteroatoms include nitrogen, oxygen, and sulfur. In certain variations, illustrative heteroatoms also include phosphorus, and selenium. As used herein, the term “cycloheteroalkyl” including heterocyclyl and heterocycle, includes a chain of atoms that includes both carbon and at least one heteroatom, such as heteroalkyl, and is optionally branched, where at least a portion of the chain is cyclic. Illustrative heteroatoms include nitrogen, oxygen, and sulfur. In certain variations, illustrative heteroatoms also include phosphorus, and selenium. Illustrative cycloheteroalkyl include, but are not limited to, tetrahydrofuryl, pyrrolidinyl, tetrahydropyranyl, piperidinyl, morpholinyl, piperazinyl, homopiperazinyl, quinuclidinyl, and the like.

As used herein, the term “aryl” includes monocyclic and polycyclic aromatic carbocyclic groups, each of which may be optionally substituted. Illustrative aromatic carbocyclic groups described herein include, but are not limited to, phenyl, naphthyl, and the like. As used herein, the term “heteroaryl” includes aromatic heterocyclic groups, each of which may be optionally substituted. Illustrative aromatic heterocyclic groups include, but are not limited to, pyridinyl, pyrimidinyl, pyrazinyl, triazinyl, tetrazinyl, quinolinyl, quinazolinyl, quinoxalinyl, thienyl, pyrazolyl, imidazolyl, oxazolyl, thiazolyl, isoxazolyl, isothiazolyl, oxadiazolyl, thiadiazolyl, triazolyl, benzimidazolyl, benzoxazolyl, benzthiazolyl, benzisoxazolyl, benzisothiazolyl, and the like.

As used herein, the term “amino” includes the group NH₂, alkylamino, and dialkylamino, where the two alkyl groups in dialkylamino may be the same or different, i.e. alkylalkylamino. Illustratively, amino includes methylamino, ethylamino, dimethylamino, methylethylamino, and the like. In addition, it is to be understood that when amino modifies or is modified by another term, such as aminoalkyl, or acylamino, the above variations of the term amino are included therein. Illustratively, aminoalkyl includes H₂N-alkyl, methylaminoalkyl, ethylaminoalkyl, dimethylaminoalkyl, methylethylaminoalkyl, and the like. Illustratively, acylamino includes acylmethylamino, acylethylamino, and the like.

As used herein, the term “amino and derivatives thereof” includes amino as described herein, and alkylamino, alkenylamino, alkynylamino, heteroalkylamino, heteroalkenylamino, heteroalkynylamino, cycloalkylamino, cycloalkenylamino, cycloheteroalkylamino, cycloheteroalkenylamino, arylamino, arylalkylamino, arylalkenylamino, arylalkynylamino, heteroarylamino, heteroarylalkylamino, heteroarylalkenylamino, heteroarylalkynylamino, acylamino, and the like, each of which is optionally substituted. The term “amino derivative” also includes urea, carbamate, and the like.

As used herein, the term “hydroxy and derivatives thereof” includes OH, and alkyloxy, alkenyloxy, alkynyloxy, heteroalkyloxy, heteroalkenyloxy, heteroalkynyloxy, cycloalkyloxy, cycloalkenyloxy, cycloheteroalkyloxy, cycloheteroalkenyloxy, aryloxy, arylalkyloxy, arylalkenyloxy, arylalkynyloxy, heteroaryloxy, heteroarylalkyloxy, heteroarylalkenyloxy, heteroarylalkynyloxy, acyloxy, and the like, each of which is optionally substituted. The term “hydroxy derivative” also includes carbamate, and the like.

As used herein, the term “thio and derivatives thereof” includes SH, and alkylthio, alkenylthio, alkynylthio, heteroalkylthio, heteroalkenylthio, heteroalkynylthio, cycloalkylthio, cycloalkenylthio, cycloheteroalkylthio, cycloheteroalkenylthio, arylthio, arylalkylthio, arylalkenylthio, arylalkynylthio, heteroarylthio, heteroarylalkylthio, heteroarylalkenylthio, heteroarylalkynylthio, acylthio, and the like, each of which is optionally substituted. The term “thio derivative” also includes thiocarbamate, and the like.

As used herein, the term “acyl” includes formyl, and alkylcarbonyl, alkenylcarbonyl, alkynylcarbonyl, heteroalkylcarbonyl, heteroalkenylcarbonyl, heteroalkynylcarbonyl, cycloalkylcarbonyl, cycloalkenylcarbonyl, cycloheteroalkylcarbonyl, cycloheteroalkenylcarbonyl, arylcarbonyl, arylalkylcarbonyl, arylalkenylcarbonyl, arylalkynylcarbonyl, heteroarylcarbonyl, heteroarylalkylcarbonyl, heteroarylalkenylcarbonyl, heteroarylalkynylcarbonyl, acylcarbonyl, and the like, each of which is optionally substituted.

As used herein, the term “carbonyl and derivatives thereof” includes the group C(O), C(S), C(NH) and substituted amino derivatives thereof.

As used herein, the term “carboxylate and derivatives thereof” includes the group CO₂H and salts thereof, and esters and amides thereof, and CN.

The term “optionally substituted” as used herein includes the replacement of hydrogen atoms with other functional groups on the radical that is optionally substituted. Such other functional groups illustratively include, but are not limited to, amino, hydroxyl, halo, thiol, alkyl, haloalkyl, heteroalkyl, aryl, arylalkyl, arylheteroalkyl, heteroaryl, heteroarylalkyl, heteroarylheteroalkyl, nitro, sulfonic acids and derivatives thereof, carboxylic acids and derivatives thereof, and the like. Illustratively, any of amino, hydroxyl, thiol, alkyl, haloalkyl, heteroalkyl, aryl, arylalkyl, arylheteroalkyl, heteroaryl, heteroarylalkyl, heteroarylheteroalkyl, and/or sulfonic acid is optionally substituted.

As used herein, the terms “optionally substituted aryl” and “optionally substituted heteroaryl” include the replacement of hydrogen atoms with other functional groups on the aryl or heteroaryl that is optionally substituted. Such other functional groups illustratively include, but are not limited to, amino, hydroxy, halo, thio, alkyl, haloalkyl, heteroalkyl, aryl, arylalkyl, arylheteroalkyl, heteroaryl, heteroarylalkyl, heteroarylheteroalkyl, nitro, sulfonic acids and derivatives thereof, carboxylic acids and derivatives thereof, and the like. Illustratively, any of amino, hydroxy, thio, alkyl, haloalkyl, heteroalkyl, aryl, arylalkyl, arylheteroalkyl, heteroaryl, heteroarylalkyl, heteroarylheteroalkyl, and/or sulfonic acid is optionally substituted.

The term “prodrug” as used herein generally refers to any compound that when administered to a biological system generates a biologically active compound as a result of one or more spontaneous chemical reaction(s), enzyme-catalyzed chemical reaction(s), and/or metabolic chemical reaction(s), or a combination thereof. In vivo, the prodrug is typically acted upon by an enzyme (such as esterases, amidases, phosphatases, and the like), simple biological chemistry, or other process in vivo to liberate or regenerate the more pharmacologically active drug. This activation may occur through the action of an endogenous host enzyme or a non-endogenous enzyme that is administered to the host preceding, following, or during administration of the prodrug. Additional details of prodrug use are described in U.S. Pat. No. 5,627,165; and Pathalk et al., Enzymic protecting group techniques in organic synthesis, Stereosel. Biocatal. 775-797 (2000). It is appreciated that the prodrug is advantageously converted to the original drug as soon as the goal, such as targeted delivery, safety, stability, and the like is achieved, followed by the subsequent rapid elimination of the released remains of the group forming the prodrug.

Prodrugs may be prepared from the compounds described herein by attaching groups that ultimately cleave in vivo to one or more functional groups present on the compound, such as —OH—, —SH, —CO₂H, —NR₂. Illustrative prodrugs include but are not limited to carboxylate esters where the group is alkyl, aryl, arylalkyl, heteroaryl, heteroarylalkyl, acyloxyalkyl, alkoxycarbonyloxyalkyl as well as esters of hydroxyl, thiol and amines where the group attached is an acyl group, an alkoxycarbonyl, aminocarbonyl, phosphate or sulfate. It is understood that the prodrugs themselves may not possess significant biological activity, but instead undergo one or more spontaneous chemical reaction(s), enzyme-catalyzed chemical reaction(s), and/or metabolic chemical reaction(s), or a combination thereof after administration in vivo to produce the compound described herein that is biologically active or is a precursor of the biologically active compound. However, it is appreciated that in some cases, the prodrug is biologically active. It is also appreciated that prodrugs may often serves to improve drug efficacy or safety through improved oral bioavailability, pharmacodynamic half-life, and the like. Prodrugs also refer to derivatives of the compounds described herein that include groups that simply mask undesirable drug properties or improve drug delivery. For example, one or more compounds described herein may exhibit an undesirable property that is advantageously blocked or minimized may become pharmacological, pharmaceutical, or pharmacokinetic barriers in clinical drug application, such as low oral drug absorption, lack of site specificity, chemical instability, toxicity, and poor patient acceptance (bad taste, odor, pain at injection site, and the like), and others. It is appreciated herein that a prodrug, or other strategy using reversible derivatives, can be useful in the optimization of the clinical application of a drug.

The term “patient” includes both human and non-human patients, such as mammals, including companion animals and other animals in captivity, such as zoo animals.

The term “therapeutically effective amount” as used herein, refers to that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the compounds and compositions described herein may be decided by the attending physician within the scope of sound medical judgment. The specific therapeutically-effective dose level for any particular patient will depend upon a variety of factors, including the disorder being treated and the severity of the disorder; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician of ordinary skill.

In addition, in those embodiments described herein drawn to combination therapy comprising administration of a chemotherapeutic agent and a small molecule therapeutic agent of the instant invention, “therapeutically effective amount” refers to that amount of the combination of agents taken together so that the combined effect elicits the desired biological or medicinal response. For example, the therapeutically effective amount of doxorubicin and a small molecule therapeutic agent of the instant invention, would be the amounts that when taken together or sequentially have a combined effect that is therapeutically effective. Further, it is appreciated that in some embodiments of such methods that include coadministration, that coadministration amount of the chemotherapeutic agent or the small molecule therapeutic agent of the instant invention when taken individually may or may not be therapeutically effective.

It is also appreciated that the therapeutically effective amount, whether referring to monotherapy or combination therapy, is advantageously selected with reference to any toxicity, or other undesirable side effect, that might occur during administration of one or more of the compounds described herein. Further, it is appreciated that the co-therapies described herein may allow for the administration of lower doses of compounds that show such toxicity, or other undesirable side effect, where those lower doses are below thresholds of toxicity or lower in the therapeutic window than would otherwise be administered in the absence of a cotherapy.

As used herein, the term “composition” generally refers to any product comprising the specified ingredients in the specified amounts, as well as any product which results, directly or indirectly, from combinations of the specified ingredients in the specified amounts. It is to be understood that the compositions described herein may be prepared from isolated compounds described herein or from salts, solutions, hydrates, solvates, and other forms of the compounds described herein. It is also to be understood that the compositions may be prepared from various amorphous, non-amorphous, partially crystalline, crystalline, and/or other morphological forms of the compounds described herein. It is also to be understood that the compositions may be prepared from various hydrates and/or solvates of the compounds described herein. Accordingly, such pharmaceutical compositions that recite compounds described herein are to be understood to include each of, or any combination of, the various morphological forms and/or solvate or hydrate forms of the compounds described herein. Illustratively, compositions may include one or more carriers, diluents, and/or excipients. The compounds described herein, or compositions containing them, may be formulated in a therapeutically effective amount in any conventional dosage forms appropriate for the methods described herein. The compounds described herein, or compositions containing them, including such formulations, may be administered by a wide variety of conventional routes for the methods described herein, and in a wide variety of dosage formats, utilizing known procedures (see generally, Remington: The Science and Practice of Pharmacy, (21^{st} ed., 2005)).

The term “administering” as used herein includes all means of introducing the compounds and compositions described herein to the patient, including, but are not limited to, oral (po), intravenous (iv), intramuscular (im), subcutaneous (sc), transdermal, inhalation, buccal, ocular, sublingual, vaginal, rectal, and the like. The compounds and compositions described herein may be administered in unit dosage forms and/or formulations containing conventional nontoxic pharmaceutically-acceptable carriers, adjuvants, and vehicles.

It is to be understood that in the methods described herein, the individual components of a co-administration, or combination can be administered by any suitable means, contemporaneously, simultaneously, sequentially, separately or in a single pharmaceutical formulation. Where the co-administered compounds or compositions are administered in separate dosage forms, the number of dosages administered per day for each compound may be the same or different. The compounds or compositions may be administered via the same or different routes of administration. The compounds or compositions may be administered according to simultaneous or alternating regimens, at the same or different times during the course of the therapy, concurrently in divided or single forms.

Illustrative routes of oral administration include tablets, capsules, elixirs, syrups, and the like.

Illustrative routes for parenteral administration include intravenous, intraarterial, intraperitoneal, epidurial, intraurethral, intrasternal, intramuscular and subcutaneous, as well as any other art recognized route of parenteral administration.

Illustrative means of parenteral administration include needle (including microneedle) injectors, needle-free injectors and infusion techniques, as well as any other means of parenteral administration recognized in the art. Parenteral formulations are typically aqueous solutions which may contain excipients such as salts, carbohydrates and buffering agents (preferably at a pH in the range from about 3 to about 9), but, for some applications, they may be more suitably formulated as a sterile non-aqueous solution or as a dried form to be used in conjunction with a suitable vehicle such as sterile, pyrogen-free water. The preparation of parenteral formulations under sterile conditions, for example, by lyophilization, may readily be accomplished using standard pharmaceutical techniques well known to those skilled in the art. Parenteral administration of a compound is illustratively performed in the form of saline solutions or with the compound incorporated into liposomes. In cases where the compound in itself is not sufficiently soluble to be dissolved, a solubilizer such as ethanol can be applied.

The dosage of each compound of the claimed combinations depends on several factors, including: the administration method, the condition to be treated, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect the dosage used.

Depending upon the disease as described herein, and the route of administration, a wide range of permissible dosages are contemplated herein, including doses falling in the range from about 1 μg/kg to about 1 g/kg. The dosages may be single or divided, and may administered according to a wide variety of protocols, including q.d., b.i.d., t.i.d., or even every other day, once a week, once a month, once a quarter, and the like. In each of these cases it is understood that the total daily, weekly, month, or quarterly dose corresponds to the therapeutically effective amounts described herein. When given locally, such as by injection near or at the site of disease, illustrative doses include those in the range from about 1 μg/kg to about 10 mg/kg, or about 0.01 mg/kg to about 10 mg/kg, or about 0.01 mg/kg to about 1 mg/kg, or about 0.1 mg/kg to about 10 mg/kg, or about 0.1 mg/kg to about 1 mg/kg. When given locally, such as by injection near the site of disease, or locally in tissues surrounding the site of disease, illustrative doses include those in the range from about 0.01 mg/kg to about 10 mg/kg, or about 0.01 mg/kg to about 1 mg/kg, or about 0.1 mg/kg to about 10 mg/kg, or about 0.1 mg/kg to about 1 mg/kg. When given systemically, such as parenterally, illustrative doses include those in the range from about 0.01 mg/kg to about 100 mg/kg, or about 0.01 mg/kg to about 10 mg/kg, or about 0.1 mg/kg to about 100 mg/kg, or about 0.1 mg/kg to about 10 mg/kg. When given systemically, such as orally, illustrative doses include those in the range from about 0.1 mg/kg to about 1000 mg/kg, or about 0.1 mg/kg to about 100 mg/kg, or about 0.1 mg/kg to about 10 mg/kg, or about 1 mg/kg to about 1000 mg/kg, or about 1 mg/kg to about 100 mg/kg, or about 1 mg/kg to about 10 mg/kg.

In another illustrative embodiment, such as when treating a disease described herein, the compound is administered parenterally locally q.d. at a dose of about 0.01 mg/kg, or about 0.05 mg/kg, or about 0.1 mg/kg, or about 0.5 mg/kg, or about 1 mg/kg, or about 5 mg/kg of body weight of the patient.

In another illustrative embodiment, such as when treating a systemic condition, the compound is administered parenterally systemically q.d. at a dose of about 0.1 mg/kg, or about 0.5 mg/kg, or about 1 mg/kg, or about 5 mg/kg, or about 10 mg/kg, or about 50 mg/kg of body weight of the patient.

In addition to the foregoing illustrative dosages and dosing protocols, it is to be understood that an effective amount of any one or a mixture of the compounds described herein can be readily determined by the attending diagnostician or physician by the use of known techniques and/or by observing results obtained under analogous circumstances. In determining the effective amount or dose, a number of factors are considered by the attending diagnostician or physician, including, but not limited to the species of mammal, including human, its size, age, and general health, the specific disease or disorder involved, the degree of or involvement or the severity of the disease or disorder, the response of the individual patient, the particular compound administered, the mode of administration, the bioavailability characteristics of the preparation administered, the dose regimen selected, the use of concomitant medication, and other relevant circumstances.

In making the pharmaceutical compositions of the compounds described herein, a therapeutically effective amount of one or more compounds in any of the various forms described herein may be mixed with one or more excipients, diluted by one or more excipients, or enclosed within such a carrier which can be in the form of a capsule, sachet, paper, or other container. Excipients may serve as a diluent, and can be solid, semi-solid, or liquid materials, which act as a vehicle, carrier or medium for the active ingredient. Thus, the formulation compositions can be in the form of tablets, pills, powders, lozenges, sachets, cachets, elixirs, suspensions, emulsions, solutions, syrups, aerosols (as a solid or in a liquid medium), ointments, soft and hard gelatin capsules, suppositories, sterile injectable solutions, and sterile packaged powders. The compositions may contain anywhere from about 0.1% to about 99.9% active ingredients, depending upon the selected dose and dosage form.

Parenteral Compositions. The pharmaceutical composition may also be administered parenterally by injection, infusion or implantation (intravenous, intramuscular, subcutaneous, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical: formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in form of a solution, a suspension, an emulsion, an infusion device, or a delivery device for implantation, or it may be presented as a dry powder to be reconstituted with water or another suitable vehicle before use. Apart from the active drug(s), the composition may include suitable parenterally acceptable carriers and/or excipients. The active drug(s) may be incorporated into microspheres, microcapsules, nanoparticles, liposomes, or the like for controlled release. Furthermore, the composition may include suspending, solubilizing, stabilizing, pH-adjusting agents, and/or dispersing agents.

As indicated above, the pharmaceutical compositions described herein may be in the form suitable for sterile injection. To prepare such a composition, the suitable active drug(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

The following examples further illustrate specific embodiments of the invention; however, the following illustrative examples should not be interpreted in any way to limit the invention.

### EXAMPLES

Compounds exemplified herein, as denoted by “AOH” numbers were obtained from AMRI (formerly Albany Medical Research, Inc.), Albany, N.Y.

Other small molecule inhibitors of caPCNA-mediated function of the invention may be prepared by conventional synthetic routes.

Identification of Compounds that Exhibit a Preferential Cytotoxicity Toward Malignant Breast Cells:

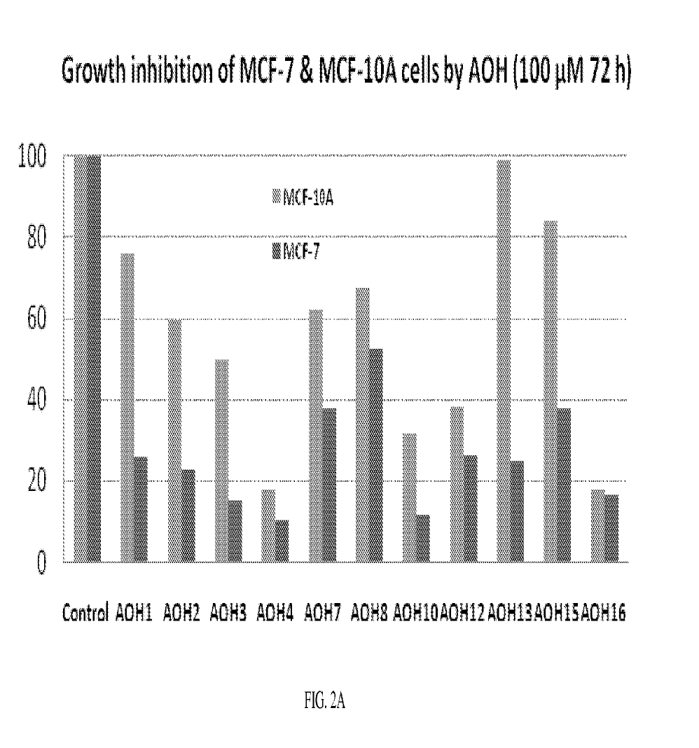

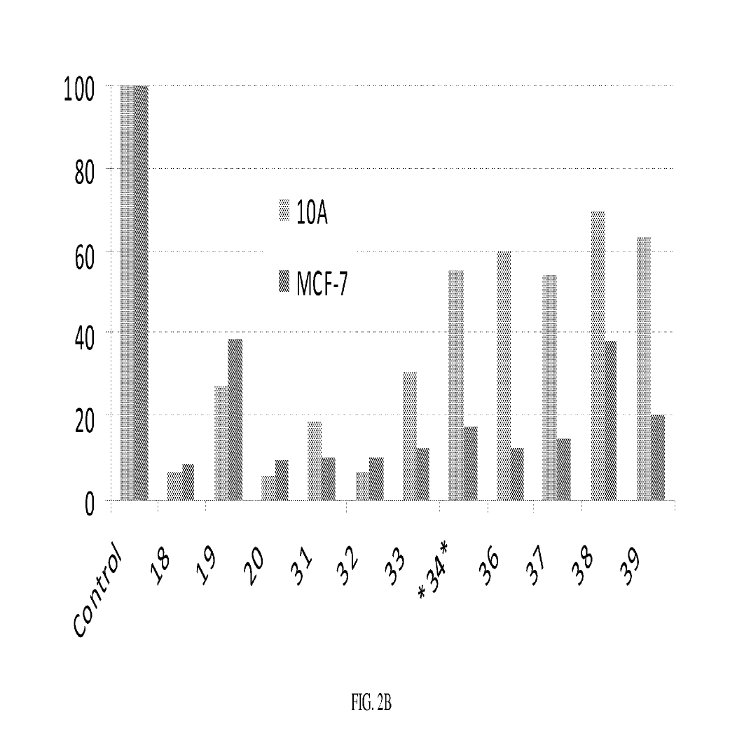

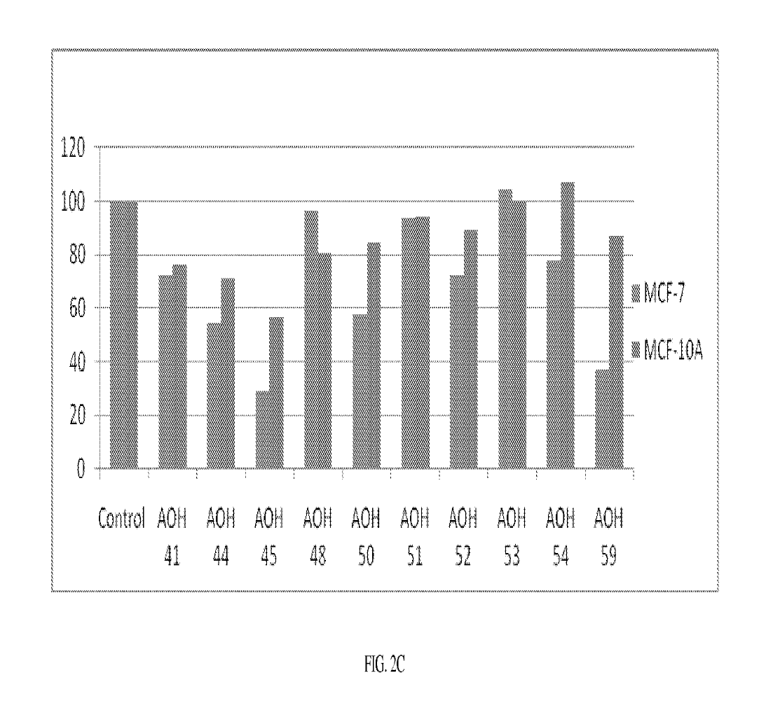

The effect that a set of in silico selected compounds had on the viability of exponentially growing cultured malignant (MCF-7) and non-malignant (MCF-10A) breast cells was determined. Exponentially growing malignant (MCF-7) and non-malignant (MCF-10A) breast cells were incubated for 72 hours with 100 μM of the indicated compounds in growth media, before cell viability was determined colorimetrically using the MTT assay. The Y-axis shows relative cell viability at the end of the incubation with the compounds. Relative viability was determined by comparison to the viability of the cell cultures incubated in the presence of PBS/DMSO (vehicle) control instead of compound. FIG. 2A-C shows that an initial cell viability screening of the in silico selected compound library identified several molecules that preferentially killed the breast cancer cells when the compounds were incubated at a concentration of 100 μM for 72 hours with each of the two cell lines. Also were identified compounds that killed: both cell types with nearly equal preference; compounds that had little or no cytotoxic effect on either cell type, and compounds that preferentially killed the non-malignant breast cells.

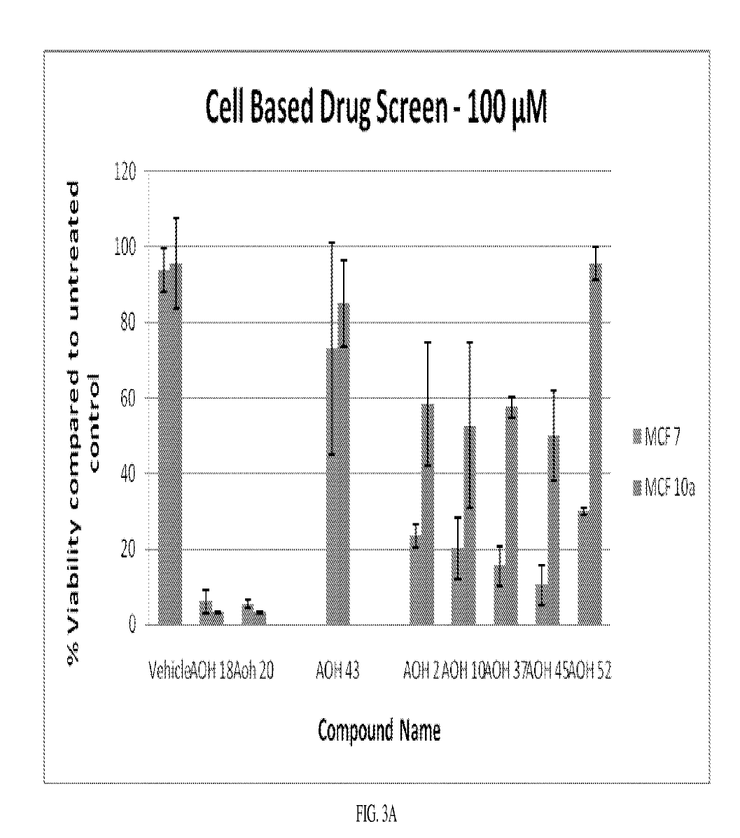

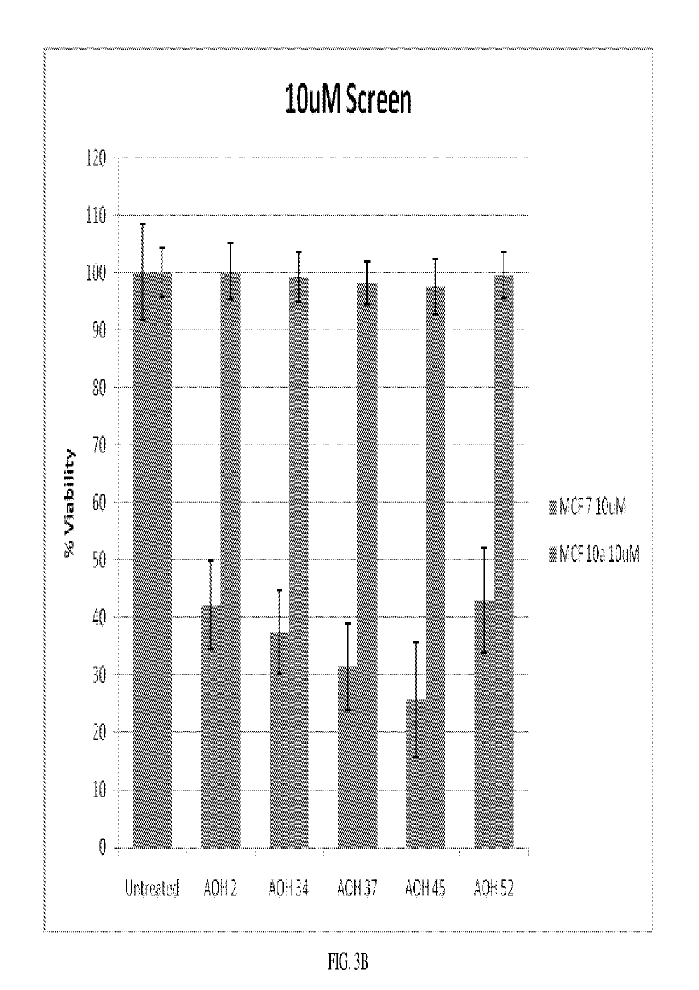

With respect to the first group, those compounds exhibiting preferential cytotoxicity toward the breast cancer cell lines were again tested in the viability assay at a 10 fold lower concentration (i.e., 10 μM), to see if any of these molecules retained their preferential cytotoxic efficacy toward the breast cancer cells. FIG. 3 shows that several of these molecules retained the ability to preferentially reduce breast cancer cell viability, while having essentially no effect on the viability of the MCF-10A cells. Together these data suggest that compounds showing preferential cytotoxicity toward the breast cancer cell lines may recognize an altered structure associated with the protein-protein interaction domain of the caPCNA molecule of breast cancer cells that is distinct from the protein-protein interaction domain of the PCNA molecule expressed by non-malignant breast cells. In addition, some of those compounds inhibiting the viability of non-malignant breast cells at 100 μM, exhibit little toxicity toward these cells when the concentration of compound is reduced. This suggests that some of the compounds used in this screening assay, which are cytotoxic to both non-malignant and malignant breast cells at the higher concentration, may in fact be selectively toxic to the breast cancer cells at lower concentrations, (e.g., AOH-18 and AOH-20).

Correlation Between the Cytotoxicity of Select AOH Compounds and their Ability to Inhibit DNA Replication.

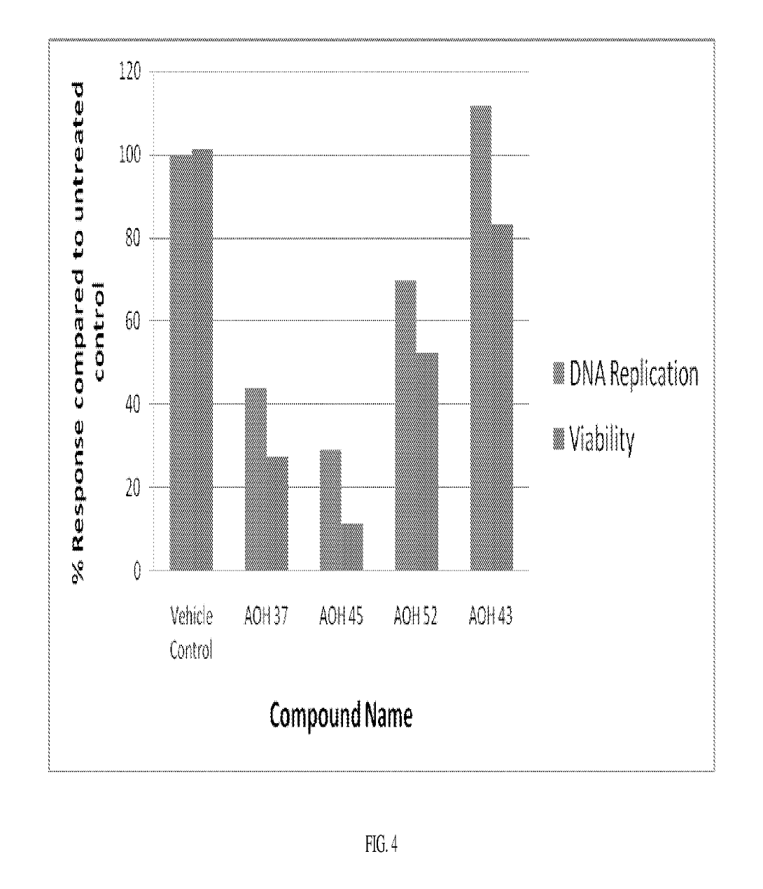

There is a precise correlation between the ability of specific AOH compounds to inhibit the SV40 origin dependent in vitro DNA replication process and MCF7 cell viability, FIG. 4. Cell viability was determined colorimetrically using the MTT assay following a 72 hour incubation with 10 μM of the compound indicated in the figure. Percent viability was determined relative to cultures containing PBS/DMSO in place of compound. In vitro DNA replication activity was determined using the standard T-antigen and SV40 origin dependent in vitro DNA replication reaction (L. Malkas et al., Biochemistry, 29, 6362-6374 (1990)), and percent inhibition was determined relative to a reaction containing PBS in place of compound. Three of those compounds exhibiting preferential cytotoxicity toward breast cancer cells were tested for their ability to inhibit the DNA synthesome mediated in vitro DNA replication process. Each of these compounds inhibited DNA replication to varying degrees, relative to a control replication reaction containing PBS/DMSO in place of compound. In vitro DNA replication reactions containing a compound which had little or no cytotoxic effect on either cell type (AOH 43) showed no inhibition of the replication reaction and only a marginal decrease in MCF7 cell viability.

### Determining the Cytotoxic Effects of the Various AOH Compounds.

The short- and long-term cytotoxic effects of each of the AOH compounds was determined essentially as described below for the caPeptide and related peptides, except that the initial cell based screening was determined by incubating cells for 72 hours with 100 μM of each compound. Compounds were classified into specific categories: 1) compounds that preferentially kill breast cancer but not non-malignant breast cells; 2) compounds that appear to kill both malignant and non-malignant breast cells without preference; 3) compounds that appear to have little or no toxicity toward either type of breast cell; and 4) compounds that appear to preferentially kill non-malignant breast cells. For those compounds falling into the first category, the short-term MTT viability assay was repeated using 10 μM of the selected AOH compound.

The Effect of caPeptide and Related Peptides on Cancer Cell Viability.

The MTT ((3-(4,5-Dimethylthiazol-2-yl)-2,5-diphenyltetrazolium bromide) assay was performed to determine the viability of cells exposed for various lengths of time to various peptide constructs containing or related to caPeptide. This assay determines the short-term affect each of the peptides has on cell viability. caPeptide's sequence is LGIPEQEY. The MTT assay was used to monitor the cytotoxic effect various caPeptides had on the viability of human MCF-7, MDA-MB436, HCC1937^{(BRCA-)} and HCC1937^{(BRCA+)} breast cancer cells. HCC1937^{(BRCA-)} cells harbor a hereditary mutation in the BRCA1 gene, that results in a non-functional gene product. These cells, are the parental wild-type to the HCC1937⁺ cells, which express a fully functional BRCA1 human transgene. The assay was performed by seeding equal numbers of cells (5×10³) into each of the wells of 96-well tissue culture plates for an overnight incubation to allow the cells to attach to the plates. In the morning, the cells in each of 3 individual wells were exposed to each of the various peptide constructs at the various concentrations indicated in the figures, for periods of 24, 48 and 72 hours. The MTT assay was then performed on the cells in each of the wells to determine the relative degree to which specific peptides were cytotoxic to each of these breast cancer cell lines. Percent viability at the end of the assay we determined colorimetrically by measuring the absorbance at 405 nm of the assay products formed in each well, averaging them, and then comparing the average absorbance of these assay products formed at each individual time period for each specific concentration of a specific peptide examined to the average absorbance of the 3 wells used to determine the viability of untreated cells exposed to PBS in place of peptide. These wells containing the PBS treated cells were assigned a relative viability of 100%, based upon the average absorbance of the reaction product formed in these wells, and the viability of the cells exposed to the various peptides under specific assay conditions was determined as a percentage of the absorbance produced by these PBS treated cells.

Long-Term Cytotoxic Effects of caPeptide and Related Peptides

A colony formation assay was used to assess the long-term cytotoxic effects the peptides had on breast cancer cell viability. Cells were pretreated with various doses of individual peptides for one hour and approximately 200 cells were plated in each of two 100 mm cell culture dishes with caPeptide-free cell culture medium for about two weeks. At the end of this period, the cells were fixed with 10% ethanol, followed by neutral formalin, and exposed to 1% Giemsa stain, prior to washing the attached stained cell colonies with one wash of 10% ethanol in PBS then 3 washes with PBS. The plates were rinsed with 10% ethanol, and air dried prior to counting.

AOH37 disrupts the binding of caPCNA with the DNA repair protein XP-G.

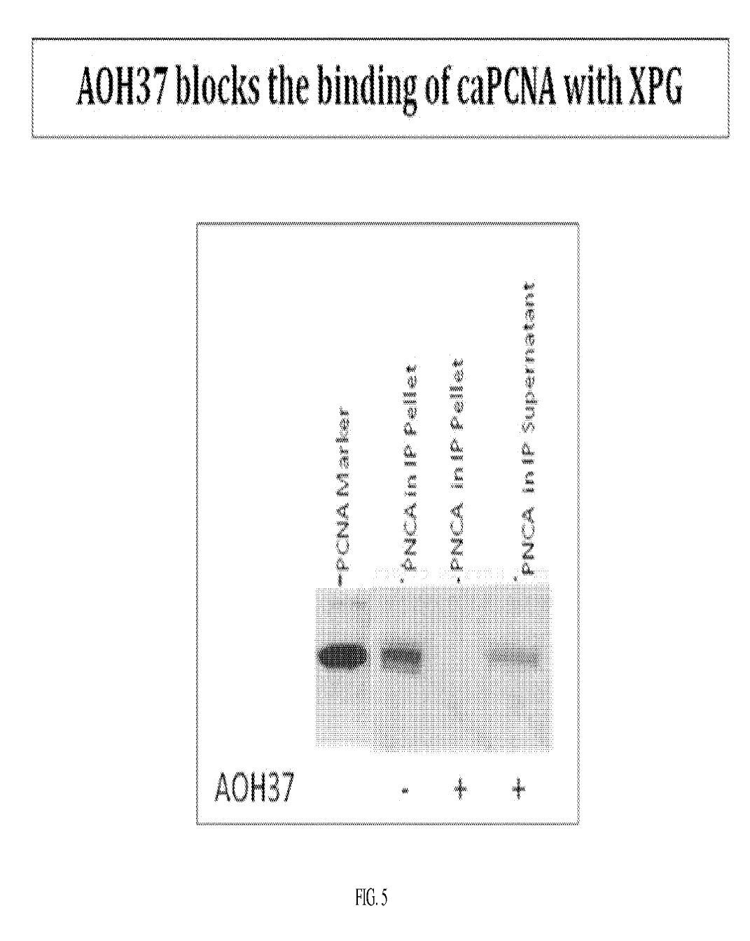

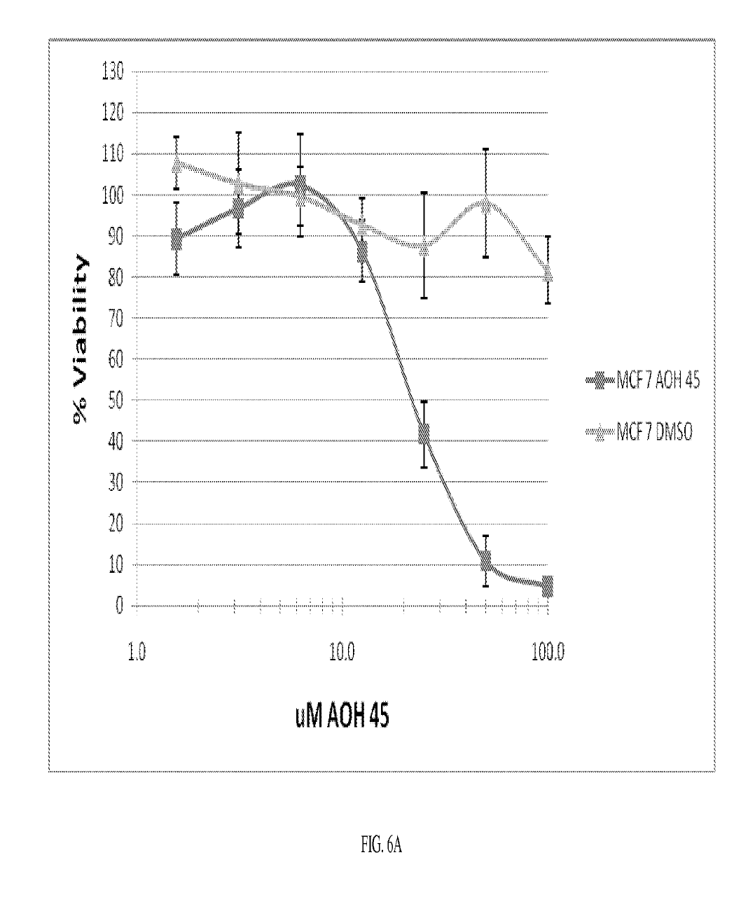

That blocking the protein-protein interaction domain of caPCNA has the potential to interfere with caPCNA's ability to interact with its naturally occurring binding partners in the cancer cell was investigated as follows. Together with PCNA, these partners perform a variety of functions that are critical to the maintenance, proliferation, and survival of the cell. One such binding partner, Xeroderma Pigmentosum protein −G is involved in the repair of specific types of DNA damage, and is a critical factor for maintaining the viability of the cancer cell. Blocking the ability of XP-G to bind to the IDCL protein-protein interaction site on caPCNA can be predicted to disrupt the DNA repair process in cells, and either promote cell killing in response to naturally occurring DNA damage, or damage promoted by DNA damaging agents such as DNA alkylating anti-cancer agents. FIG. 5, lane 2 shows that PCNA is present in the anti-XPG antibody bound immunoprecipitate when AOH 37 is not pre-incubated with the cell extracts, but is absent from the immunoprecipitae when the extract is pre-treated with AOH37 (lane 3). Lane 4 shows that PCNA can be found in the supernatant fraction of the immunoprecipitation reaction when the cell lysate is pre-incubated with AOH37. These data clearly indicate that XP-G and PCNA are bound to one another in the MCF7 cells, and that pre-incubation with a compound selectively targeting the interaction domain disrupts the XPG-PCNA couple; releasing PCNA into the supernatant of the immunoprecipitate reaction. This is the first evidence directly linking the ability of any of the in silico derived compounds to bind to the protein-protein interaction domain of the IDCL loop of PCNA, and disrupt a specific protein-protein interaction.

### Effect of AOH-45 on Malignant and Non-Malignant Cell Viability.

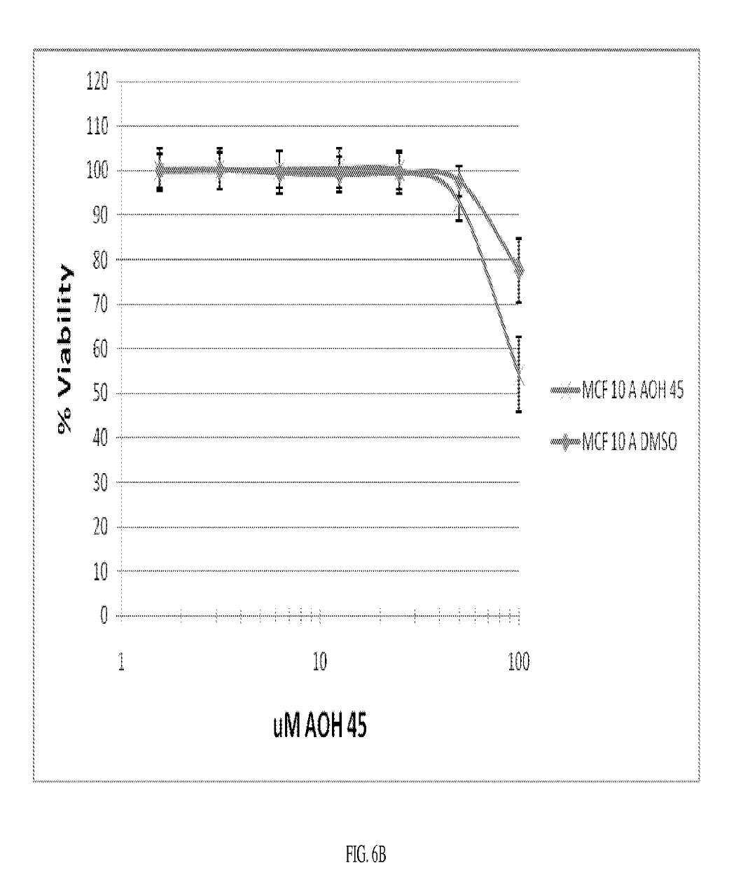

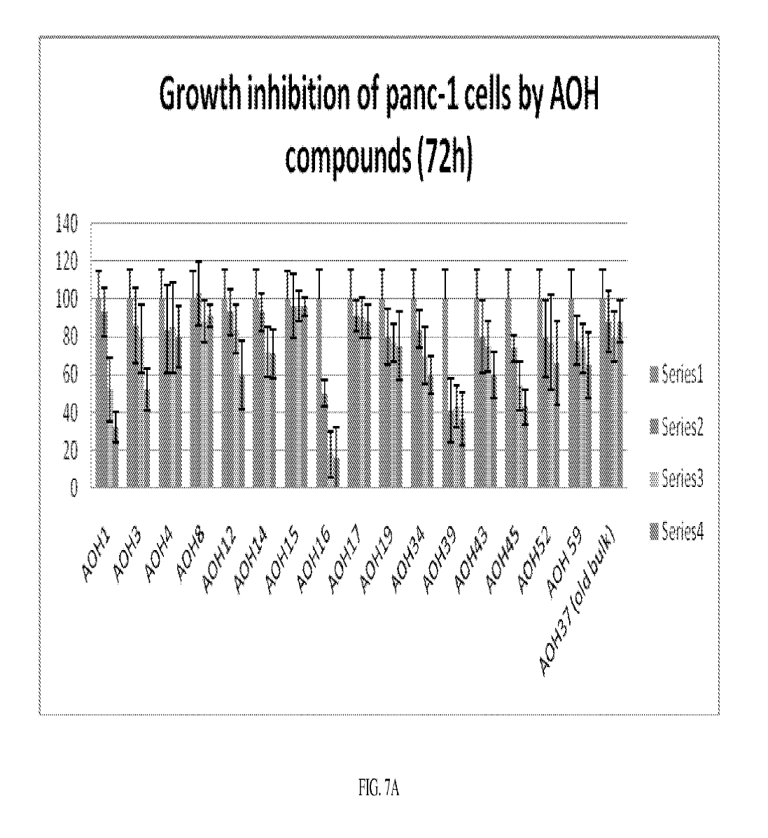

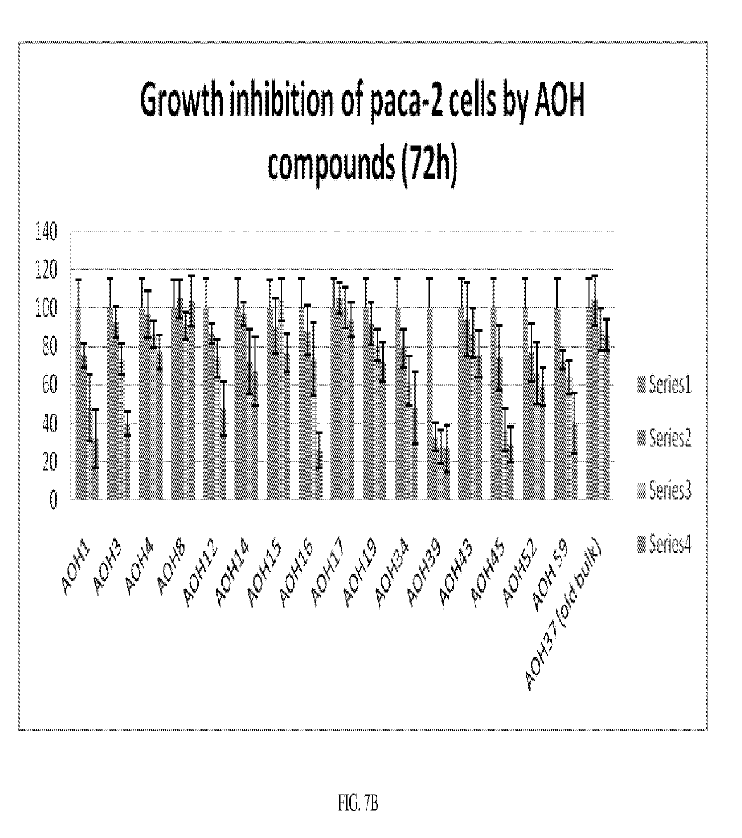

AOH-45 exhibits a profound differential cytotoxicity toward breast cancer cells. Because the compounds screened in our cell culture assay are dissolved in 100% DMSO, we add differing amounts of DMSO to the cell culture media, corresponding to the amount of DMSO added along with compound, while performing the screening assay. To better understand the contribution DMSO maybe making to the cytotoxic effects noted for each compound we examined, we determined the relative cytotoxicity at each DMSO concentration present in the cell culture resulting from the addition of compound to the cell culture dishes. Our results are shown in FIG. 6. For the MCF7 cells, AOH-45 reduced cell viability beginning at ˜10 μM, and exhibited an LC₅₀=20 μM and an LC₉₀=50 μM. In contrast exposing the MCF10A cells to AOH-45 below 50 μM or to DMSO (below 0.5%) resulted in only a minimal decrease in viability (<5-10%). DMSO only began to significantly decrease MCF10A cell viability (20%) when its concentration in the growth media approached 1%.

### Relative Cytotoxicity of AOH Compounds Toward Pancreatic Cancer

Pancreatic cancer cells appeared to be differentially sensitive to the cytotoxic effects of each of the compounds indicated. AOH-4, 8, 15, 17, “old” 37, 43, 19 showed little overall cytotoxicity toward these pancreatic cancer lines regardless of the concentration of compound in the tissue culture media.

In contrast, pancreatic cancer cells exhibit a concentration dependent cytotoxicity toward AOH-1, 3, 12, 14, 16, 19, 34, 39, 43, 45, 52, and 59. Of these, AOH-1, 3, 12, 16, 34, 45 and 59 exhibit a concentration dependent cytotoxicity, while cells appear to be especially sensitive to AOH-39 even at 12.5 μM. Compounds exhibiting strong concentration dependent killing were also screened for cytotoxicity toward normal proliferating cells (peripheral blood mononucleocytes (i.e., PBMC's)).

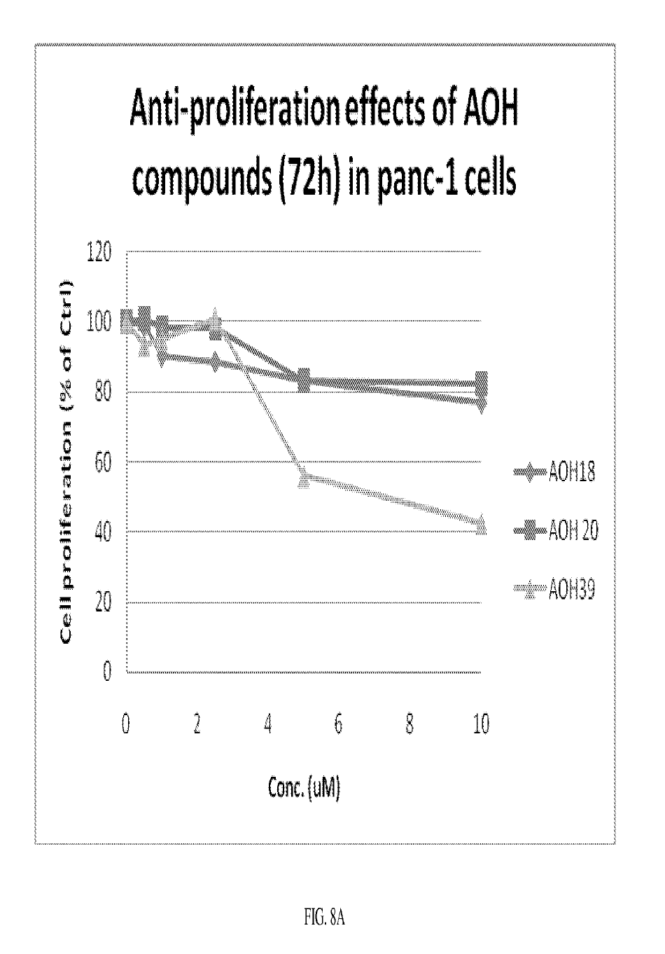

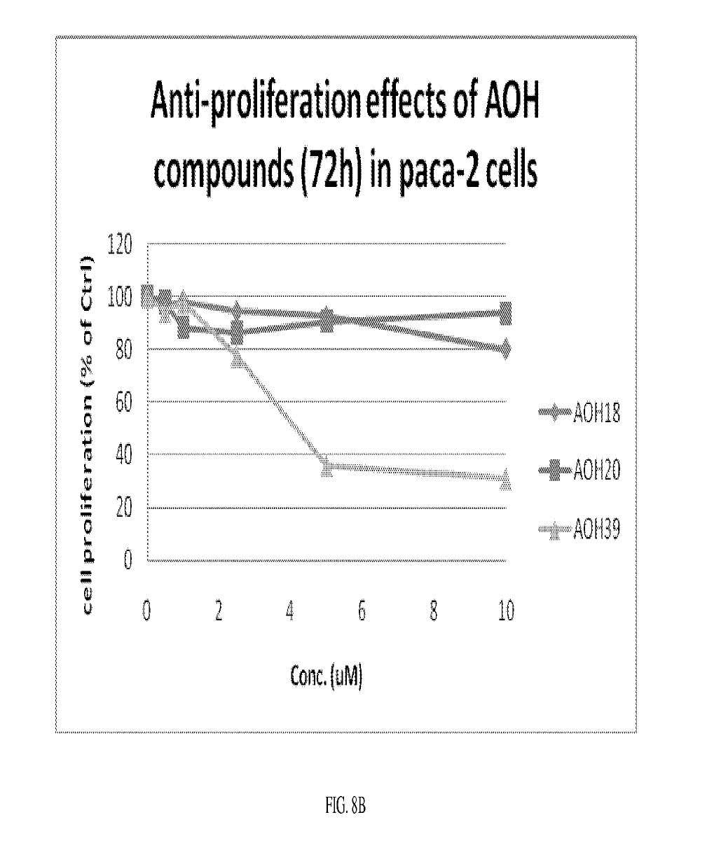

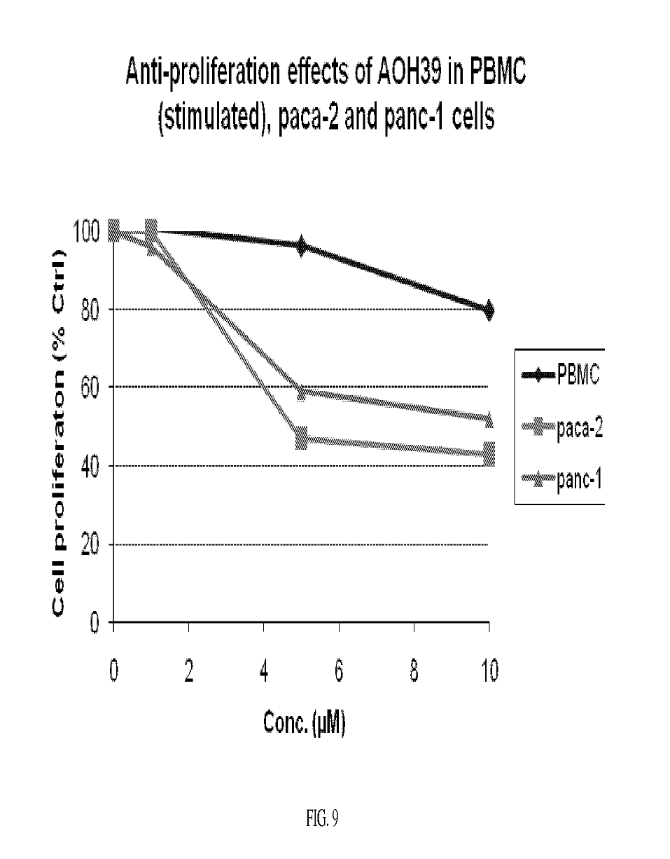

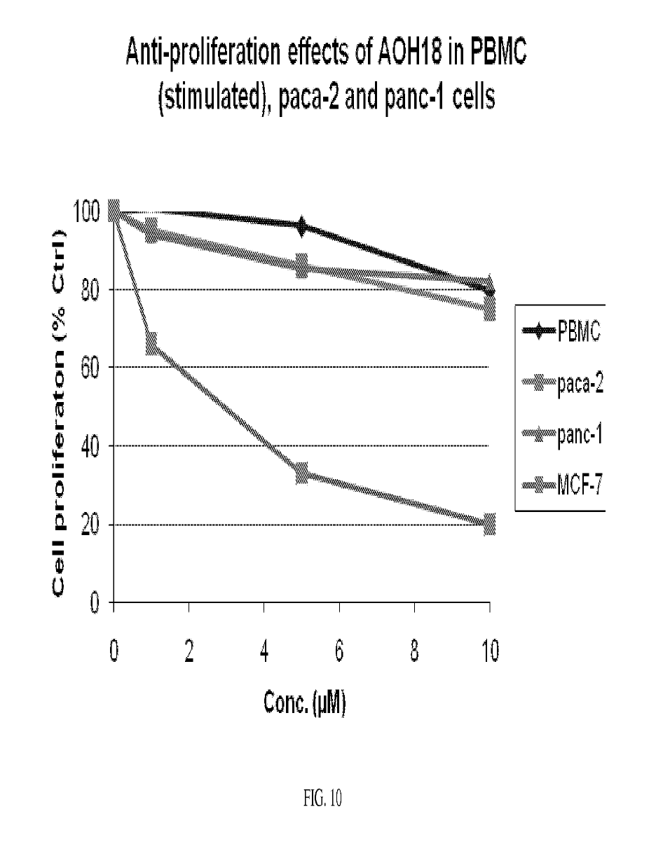

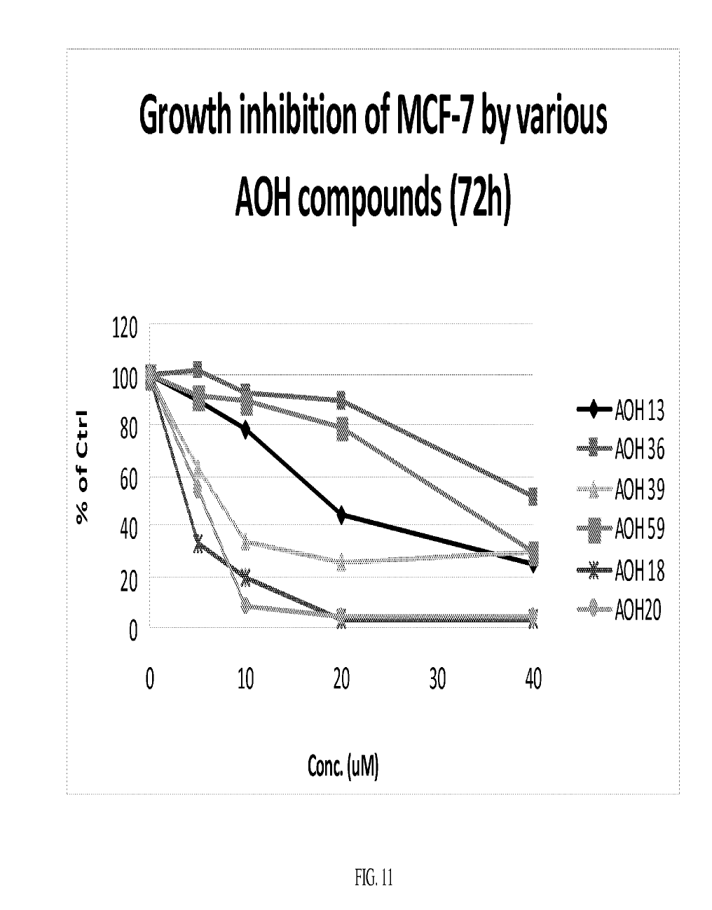

Two additional compounds (AOH-18 and AOH-20), were also examined in the pancreatic cancer cell lines. Neither AOH-18 or AOH-20 appeared to be as cytotoxic as AOH-39 toward either of the two cell lines. AOH-39 exhibited an LC₅₀ of 5 μM in the Panc-1 cells, and 4 μM in the Paca-2 cells; while the LC₅₀ for both AOH-18 and AOH20 was not achieved over the concentration range tested, FIG. 8. These and other data suggest that AOH-39 may be an effective pancreatic cancer chemotherapeutic agent capable of selectively killing pancreatic cancer cells at relatively low effective concentrations, while exhibiting little cytotoxicity toward normal proliferating cells (peripheral blood mononucleocytes), FIG. 9. AOH-39 did not inhibit PBMC cell viability at the LC₅₀ for Paca-2 cells, and only slightly reduced viability of the Panc-1 cells.

Differential Response of Breast Cancer vs. Pancreatic Cancer Cell Viability to AOH-18.

Unlike pancreatic cancer cells, the breast cancer cell line MCF-7 exhibited a profound sensitivity to AOH-18. This was in sharp contrast to the sensitivity of stimulated PBMC, paca-2, and panc-1 cells. MCF-7 cells exhibited an LC₅₀ for AOH-18 of ˜3 μM, while both pancreatic cell lines and the stimulated PBMC's exhibited little or no loss in viability at this same concentration of compound.

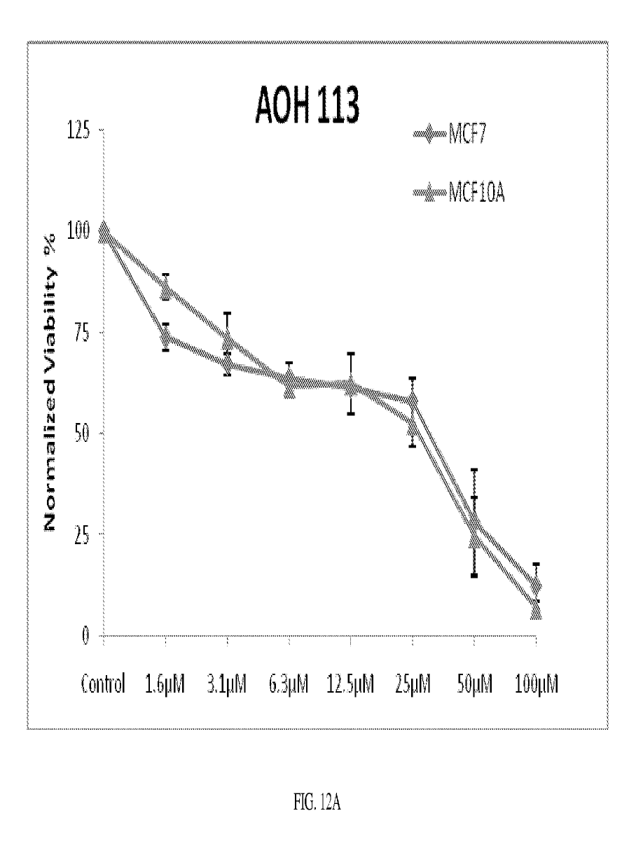

In addition, MCF-7 cell viability appeared to be differentially sensitive to the cytotoxic effect of several of the compounds (AOH-13, -18, -20, -36, -39, and 59), with LC₅₀ values ranging from ˜3 through 40 μM, FIG. 11. The MCF-7 cells were most sensitive to AOH-18, followed by AOH-20, AOH-39, AOH-13, AOH-59, and AOH-36. Our data indicate that unlike pancreatic cancer cells, which are very sensitive to the cytotoxic effects of AOH-39, but not AOH-18, MCF-7 cells are most sensitive to the cytotoxic effects of AOH-18.

Compounds which Bind to the Protein-Protein Interaction Domain of caPCNA Binding Partner Fen1.

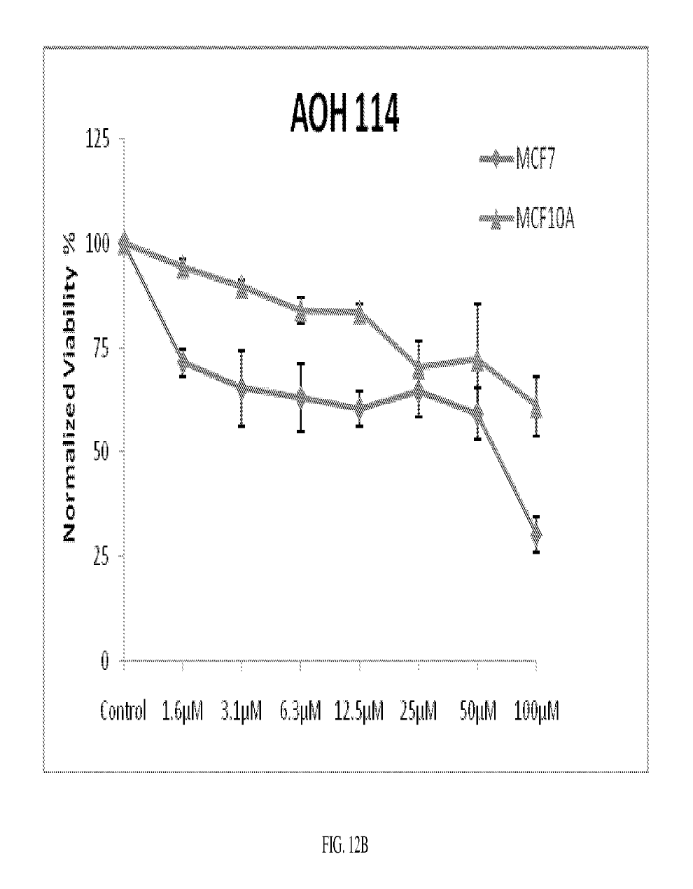

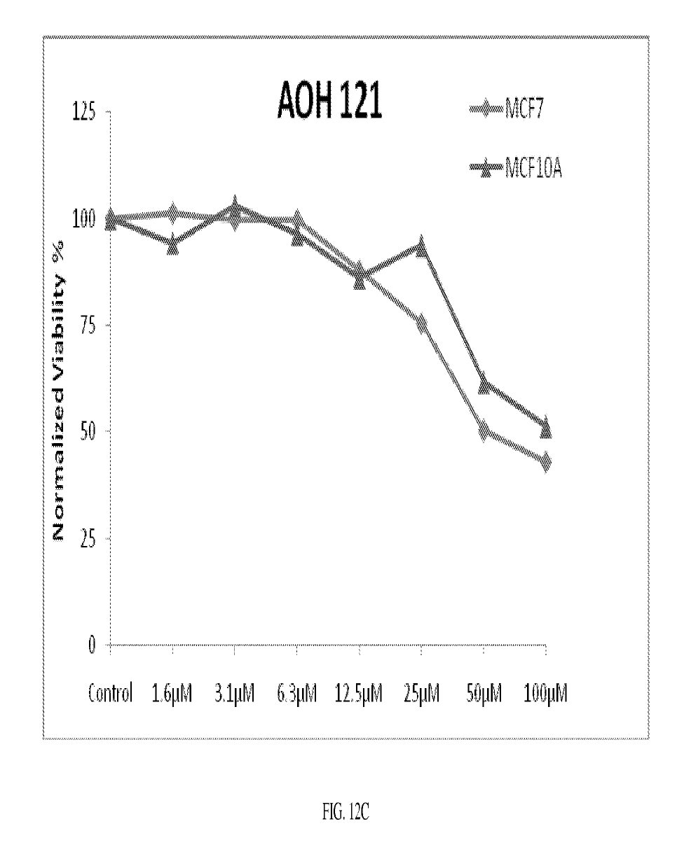

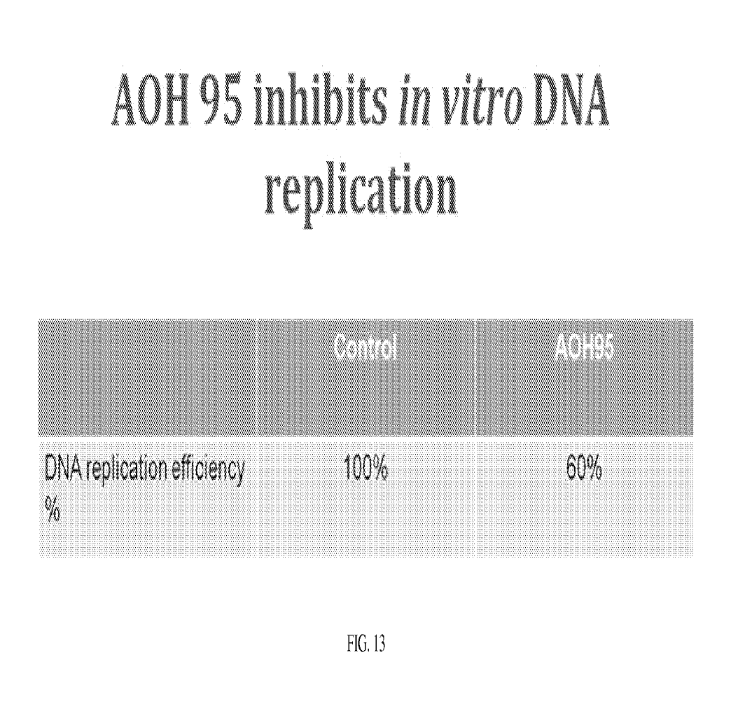

The effect that a set of in silico selected compounds had on the viability of exponentially growing cultured malignant (MCF-7) and non-malignant (MCF-10A) breast cells was determined by testing for their affect on the viability of malignant and non-malignant breast cells, FIG. 12. The LC₅₀ of AOH-94, AOH-95, and AOH-120 were determined for each cell type and compared to one another for the MCF7 cells. AOH95 has an LC₅₀ of ˜20 M, which is nearly half of that of AOH-94 and AOH-120. In addition, at 25 μM AOH-95 had only a marginal effect on the viability of the non-malignant breast cells.

### AOH95 Inhibits the DNA Synthetic Process.

Because of the critical role Fen1 plays in the DNA replication process, we examined the possibility that the preferential killing of breast cancer cells by AOH95 was mediated, at least in part, by the compound's ability to inhibit the DNA synthetic process. AOH-95 at its LC₅₀ value was pre-incubated with the DNA synthesome fraction isolated from MCF7 breast cancer cells prior to initiating the DNA synthetic reaction. The amount of radiolabeled nucleotide triphosphate incorporated into nascent DNA by the DNA synthesome following pre-treatment with AOH-95 was determined as described previously (Malkas et al., 1990, Biochemistry), and compared to the amount of radiolabeled nucleotide triphosphate incorporated by untreated DNA synthesome. The results of this study presented in FIG. 13 indicate that AOH-95 inhibits the in vitro DNA replication activity of the isolated MCF7 breast cancer cell DNA synthesome by 40% relative to the untreated DNA synthesome from these cells in our assay. Our finding is consistent with AOH95 disrupting the DNA synthetic activity of the breast cancer cell DNA synthesome through inhibition of Fen1, and together with the cell viability data, suggests that AOH95 may act selectively to preferentially kill breast cancer cells by inhibiting the activity of the breast cancer cell DNA synthetic process.

What is claimed is:

1. A method of reducing cellular proliferation of malignant cells that express a cancer specific isoform of proliferating cell nuclear antigen (caPCNA) in a patient in need thereof, comprising administering a therapeutically effective amount of a compound of the formula 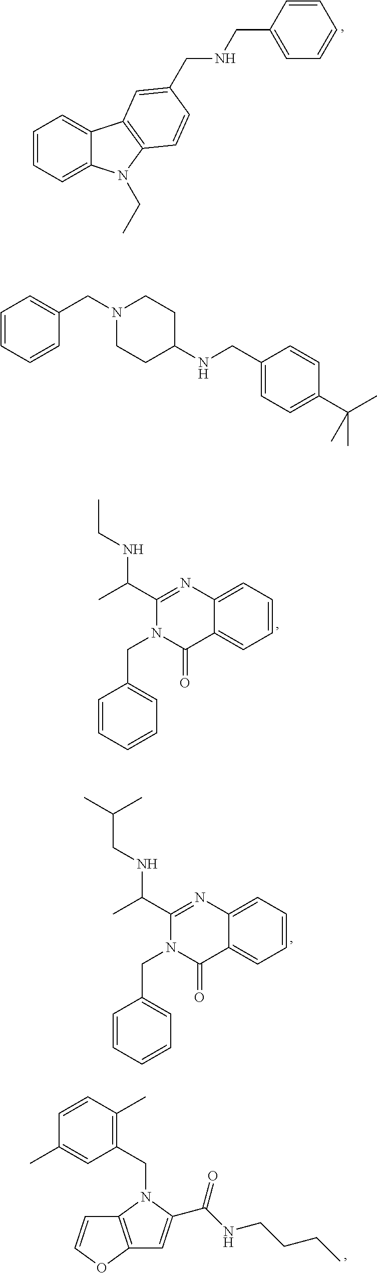 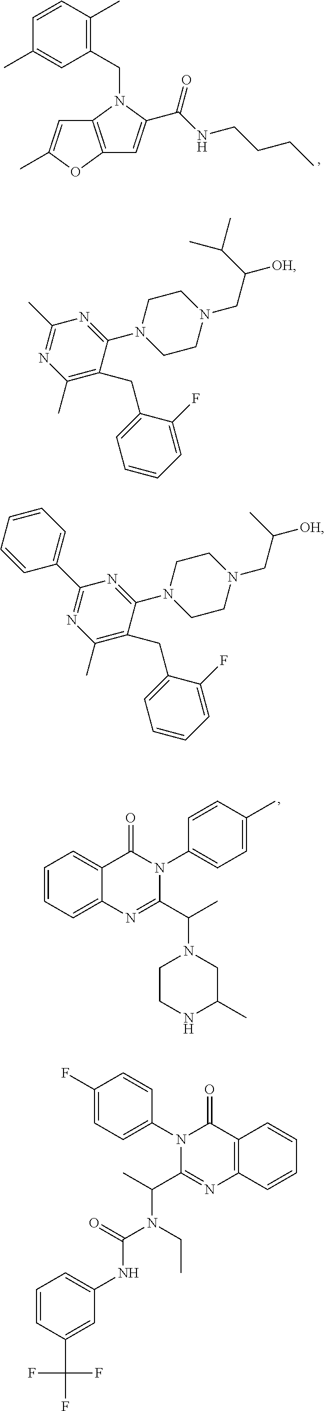 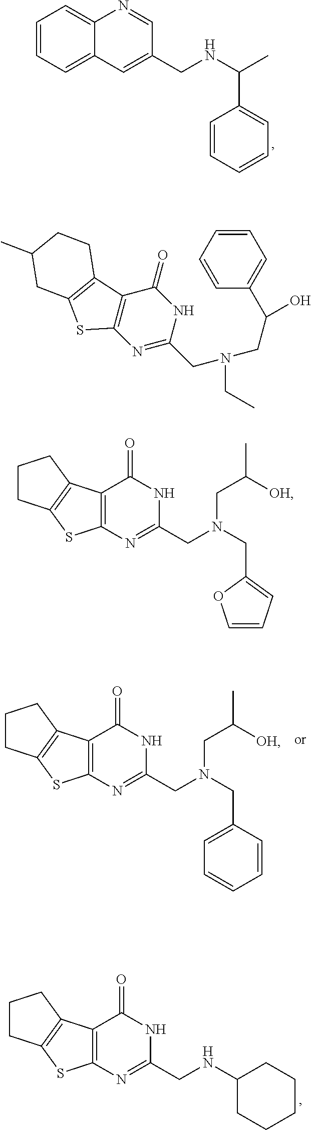
or a substituted derivative thereof, or a pharmaceutically acceptable salt thereof.

2. Use of a compound as described in claim 1 or a substituted derivative thereof, or a pharmaceutically acceptable salt thereof, for reducing cellular proliferation of malignant cells that express a cancer specific isoform of proliferating cell nuclear antigen (caPCNA).

3. A pharmaceutical composition comprising a compound as described in claim 1 or a substituted derivative thereof, or a pharmaceutically acceptable salt thereof, and further comprising one or more carriers, diluents, or excipients, or a combination thereof.

4. The method, use or composition of claim 1 wherein the compound is 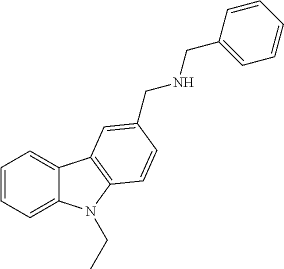 [SMILES: CCn1c2ccccc2c2cc(CNCc3ccccc3)ccc21]
or a substituted derivative thereof, or a pharmaceutically acceptable salt thereof.

5. The method, use or composition of claim 1 wherein the compound is 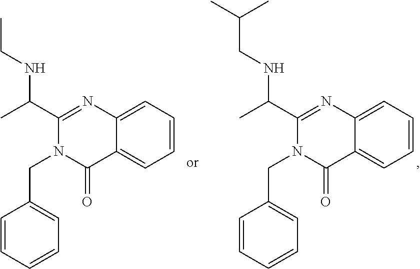 [SMILES: CCNC(C)c1nc2ccccc2c(=O)n1Cc1ccccc1 ; CC(C)CNC(C)c1nc2ccccc2c(=O)n1Cc1ccccc1]
or a substituted derivative thereof, or a pharmaceutically acceptable salt thereof.

6. The method, use or composition of claim 1 wherein the compound is 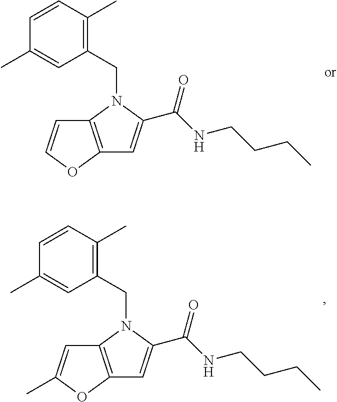
or a substituted derivative thereof, or a pharmaceutically acceptable salt thereof.

7. The method, use or composition of claim 1 wherein the compound is 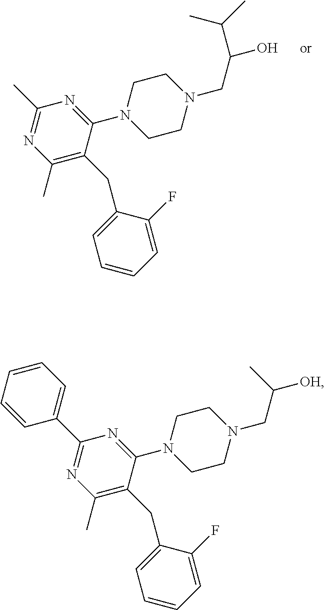 [SMILES: Cc1nc(C)c(Cc2ccccc2F)c(N2CCN(CC(O)C(C)C)CC2)n1 ; Cc1nc(-c2ccccc2)nc(N2CCN(CC(C)O)CC2)c1Cc1ccccc1F]
or a substituted derivative thereof, or a pharmaceutically acceptable salt thereof.

8. The method, use or composition of claim 1 wherein the compound is 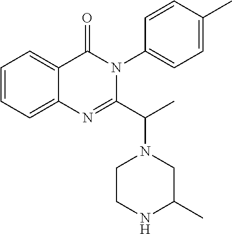 [SMILES: Cc1ccc(-n2c(C(C)N3CCNC(C)C3)nc3ccccc3c2=O)cc1]
or a substituted derivative thereof, or a pharmaceutically acceptable salt thereof.

9. The method, use or composition of claim 1 wherein the compound is 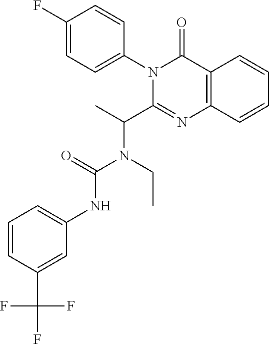 [SMILES: CCN(C(=O)Nc1cccc(C(F)(F)F)c1)C(C)c1nc2ccccc2c(=O)n1-c1ccc(F)cc1]
or a substituted derivative thereof, or a pharmaceutically acceptable salt thereof.

10. The method, use or composition of claim 1 wherein the compound is 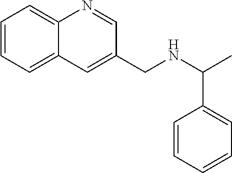 [SMILES: CC(NCc1cnc2ccccc2c1)c1ccccc1]
or a substituted derivative thereof, or a pharmaceutically acceptable salt thereof.

11. The method, use or composition of claim 1 wherein the compound is 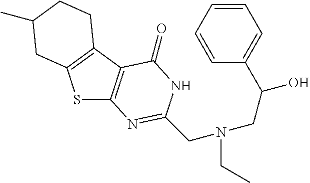 [SMILES: CCN(Cc1nc2sc3c(c2c(=O)[nH]1)CCC(C)C3)CC(O)c1ccccc1]
or a substituted derivative thereof, or a pharmaceutically acceptable salt thereof.

12. The method, use or composition of claim 1 wherein the compound is 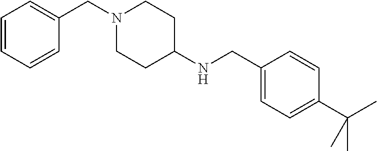 [SMILES: CC(C)(C)c1ccc(CNC2CCN(Cc3ccccc3)CC2)cc1]
or a substituted derivative thereof, or a pharmaceutically acceptable salt thereof.

13. The method, use or composition of claim 1 wherein the cancer is breast cancer.

14. The method, use or composition of claim 1 wherein the cancer is pancreatic cancer.

15. The method or use of claim 2 wherein the use is to augment another chemotherapeutic method.

16. A pharmaceutical composition comprising a compound as described in claim 1 and a further chemotherapeutic agent.
